SEQUENCE LISTING (64 sequences)

SEQ ID NO: 1 - protein, 6 aa, Homo sapiens
TVLTVV

SEQ ID NO: 2 - protein, 11 aa, Homo sapiens
LLLLLTVLTVV

SEQ ID NO: 3 - protein, 21 aa, Homo sapiens
MTPGTQSPFFLLLLLTVLTVV

SEQ ID NO: 4 - protein, 10 aa, Homo sapiens
KKKKTVLTVV

SEQ ID NO: 5 - protein, 15 aa, Homo sapiens
KKKKLLLLLTVLTVV

SEQ ID NO: 6 - protein, 25 aa, Homo sapiens
KKKKMTPGTQSPFFLLLLLTVLTVV

SEQ ID NO: 7 - protein, 6 aa, Homo sapiens
  MISC_FEATURE (1)..(1): polyethylene glycol (PEG) backbone modification
TVLTVV

SEQ ID NO: 8 - protein, 11 aa, Homo sapiens
  MISC_FEATURE (1)..(1): polyethylene glycol (PEG) backbone modification
LLLLLTVLTVV

SEQ ID NO: 9 - protein, 21 aa, Homo sapiens
  MISC_FEATURE (1)..(1): polyethylene glycol (PEG) backbone modification
MTPGTQSPFFLLLLLTVLTVV

SEQ ID NO: 10 - protein, 10 aa, Homo sapiens
  MISC_FEATURE (4)..(5): polyethylene glycol (PEG) backbone modification between residues 4 and 5
KKKKTVLTVV

SEQ ID NO: 11 - protein, 15 aa, Homo sapiens
  MISC_FEATURE (4)..(5): polyethylene glycol (PEG) backbone modification between residues 4 and 5
KKKKLLLLLTVLTVV

SEQ ID NO: 12 - protein, 25 aa, Homo sapiens
  MISC_FEATURE (4)..(5): polyethylene glycol (PEG) backbone modification between residues 4 and 5
KKKKMTPGTQSPFFLLLLLTVLTVV

SEQ ID NO: 13 - protein, 6 aa, Homo sapiens
  MISC_FEATURE (1)..(1): Alexa Fluor 594 backbone modification
TVLTVV

SEQ ID NO: 14 - protein, 11 aa, Homo sapiens
  MISC_FEATURE (1)..(1): Alexa Fluor 594 backbone modification
LLLLLTVLTVV

SEQ ID NO: 15 - protein, 21 aa, Homo sapiens
  MISC_FEATURE (1)..(1): Alexa Fluor 594 backbone modification
MTPGTQSPFFLLLLLTVLTVV

SEQ ID NO: 16 - protein, 5 aa, Homo sapiens
  MISC_FEATURE (1)..(1): Alexa Fluor 594 attached to polyethylene glycol (PEG) backbone modification
TVLTV

SEQ ID NO: 17 - protein, 11 aa, Homo sapiens
  MISC_FEATURE (1)..(1): Alexa Fluor 594 attached to polyethylene glycol (PEG) backbone modification
LLLLLTVLTVV

SEQ ID NO: 18 - protein, 21 aa, Homo sapiens
  MISC_FEATURE (1)..(1): Alexa Fluor 594 attached to polyethylene glycol (PEG) backbone modification
MTPGTQSPFFLLLLLTVLTVV

SEQ ID NO: 19 - protein, 6 aa, Homo sapiens
  MISC_FEATURE (1)..(1): Alexa Fluor 594 attached to polyethylene glycol (PEG) backbone modification
TVLTVV

SEQ ID NO: 20 - protein, 15 aa, Homo sapiens
  MISC_FEATURE (1)..(1): Alexa Fluor 594 backbone modification
  MISC_FEATURE (4)..(5): polyethylene glycol (PEG) backbone modification between residues 4 and 5
KKKKLLLLLTVLTVV

SEQ ID NO: 21 - protein, 25 aa, Homo sapiens
  MISC_FEATURE (1)..(1): Alexa Fluor 594 backbone modification
  MISC_FEATURE (4)..(5): polyethylene glycol (PEG) backbone modification between residues 4 and 5
KKKKMTPGTQSPFFLLLLLTVLTVV

SEQ ID NO: 22 - protein, 28 aa, Homo sapiens
KKKKGGGMTPGTQSPFFLLLLLTVLTVV

SEQ ID NO: 23 - protein, 27 aa, Homo sapiens
KKKKGGGTPGTQSPFFLLLLLTVLTVV

SEQ ID NO: 24 - protein, 10 aa, Homo sapiens
RRRRTVLTVV

SEQ ID NO: 25 - protein, 15 aa, Homo sapiens
RRRRLLLLLTVLTVV

SEQ ID NO: 26 - protein, 10 aa, Homo sapiens
HHHHTVLTVV

SEQ ID NO: 27 - protein, 15 aa, Homo sapiens
HHHHLLLLLTVLTVV

SEQ ID NO: 28 - protein, 10 aa, Homo sapiens
NNNNTVLTVV

SEQ ID NO: 29 - protein, 15 aa, Homo sapiens
NNNNLLLLLTVLTVV

SEQ ID NO: 30 - protein, 20 aa, Homo sapiens
TPGTQSPFFLLLLLTVLTVV

SEQ ID NO: 31 - protein, 24 aa, Homo sapiens
KKKKTPGTQSPFFLLLLLTVLTVV

SEQ ID NO: 32 - protein, 24 aa, Homo sapiens
  MISC_FEATURE (1)..(1): (Gd)DOTA - metal label backbone modification
  MISC_FEATURE (4)..(5): polyethylene glycol (PEG) backbone modification between residues 4 and 5
  MISC_FEATURE (14)..(18): photoleucine
KKKKTPGTQSPFFLLLLLTVLTVV

SEQ ID NO: 33 - protein, 24 aa, Homo sapiens
  MISC_FEATURE (1)..(1): (Gd)DOTA - metal label backbone modification
  MISC_FEATURE (4)..(5): polyethylene glycol (PEG) backbone modification between residues 4 and 5
  MISC_FEATURE (14)..(18): photoleucine
KKKKTPGTQSPFFLLLLLTVLTVV

SEQ ID NO: 34 - protein, 24 aa, Homo sapiens
  MISC_FEATURE (1)..(1): biotin tag backbone modification
  MISC_FEATURE (4)..(5): polyethylene glycol (PEG) backbone modification between residues 4 and 5
KKKKTPGTQSPFFLLLLLTVLTVV

SEQ ID NO: 35 - protein, 24 aa, Homo sapiens
  MISC_FEATURE (1)..(1): biotin tag backbone modification
  MISC_FEATURE (4)..(5): polyethylene glycol (PEG) backbone modification between residues 4 and 5
  MISC_FEATURE (14)..(18): photoleucine
KKKKTPGTQSPFFLLLLLTVLTVV

SEQ ID NO: 36 - protein, 23 aa, Homo sapiens
KKKKPGTQSPFFLLLLLTVLTVV

SEQ ID NO: 37 - protein, 22 aa, Homo sapiens
KKKKGTQSPFFLLLLLTVLTVV

SEQ ID NO: 38 - protein, 21 aa, Homo sapiens
KKKKTQSPFFLLLLLTVLTVV

SEQ ID NO: 39 - protein, 20 aa, Homo sapiens
KKKKQSPFFLLLLLTVLTVV

SEQ ID NO: 40 - protein, 19 aa, Homo sapiens
KKKKSPFFLLLLLTVLTVV

SEQ ID NO: 41 - protein, 18 aa, Homo sapiens
KKKKPFFLLLLLTVLTVV

SEQ ID NO: 42 - protein, 17 aa, Homo sapiens
KKKKFFLLLLLTVLTVV

SEQ ID NO: 43 - protein, 16 aa, Homo sapiens
KKKKFLLLLLTVLTVV

SEQ ID NO: 44 - protein, 1259 aa, Homo sapiens
KKKKMTPGTQSPFFLLLLLTVLTVVTGSGHASSTPGGEKETSATQRSSVPSSTEKNAVSMTSSVLSSHSPGSGSSTTQGQDVTLAPATEPASGSAATWGQDVTSVPVTRPALGSTTPPAHDVTSAPDNKPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDNRPALGSTAPPVHNVTSASGSASGSASTLVHNGTSARATTTPASKSTPFSIPSHHSDTPTTLASHSTKTDASSTHHSSVPPLTSSNHSTSPQLSTGVSFFFLSFHISNLQFNSSLEDPSTDYYQELQRDISEMFLQIYKQGGFLGLSNIKFRPGSVVVQLTLAFREGTINVHDVETQFNQYKTEAASRYNLTISDVSVSDVPFPFSAQSGAGVPGWGIALLVLVCVLVALAIVYLIALAVCQCRRKNYGQLDIFPARDTYHPMSEYPTYHTHGRYVPPSSTDRSPYEKVSAGNGGSSLSYTNPAVAATSANL

SEQ ID NO: 45 - protein, 1255 aa, Homo sapiens
  MISC_FEATURE (1)..(1): polyethylene glycol (PEG) backbone modification
MTPGTQSPFFLLLLLTVLTVVTGSGHASSTPGGEKETSATQRSSVPSSTEKNAVSMTSSVLSSHSPGSGSSTTQGQDVTLAPATEPASGSAATWGQDVTSVPVTRPALGSTTPPAHDVTSAPDNKPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDNRPALGSTAPPVHNVTSASGSASGSASTLVHNGTSARATTTPASKSTPFSIPSHHSDTPTTLASHSTKTDASSTHHSSVPPLTSSNHSTSPQLSTGVSFFFLSFHISNLQFNSSLEDPSTDYYQELQRDISEMFLQIYKQGGFLGLSNIKFRPGSVVVQLTLAFREGTINVHDVETQFNQYKTEAASRYNLTISDVSVSDVPFPFSAQSGAGVPGWGIALLVLVCVLVALAIVYLIALAVCQCRRKNYGQLDIFPARDTYHPMSEYPTYHTHGRYVPPSSTDRSPYEKVSAGNGGSSLSYTNPAVAATSANL

SEQ ID NO: 46 - protein, 1259 aa, Homo sapiens; identical to SEQ ID NO: 44
  MISC_FEATURE (4)..(5): polyethylene glycol (PEG) backbone modification

SEQ ID NO: 47 - protein, 1255 aa, Homo sapiens; identical to SEQ ID NO: 45
  MISC_FEATURE (1)..(1): Alexa Fluor 594 backbone modification

SEQ ID NO: 48 - protein, 1255 aa, Homo sapiens; identical to SEQ ID NO: 45
  MISC_FEATURE (1)..(1): Alexa Fluor 594 attached to polyethylene glycol (PEG) bcakbone modification

SEQ ID NO: 49 - protein, 1259 aa, Homo sapiens; identical to SEQ ID NO: 44
  MISC_FEATURE (1)..(1): Alexa Fluor 594 backbone modification
  MISC_FEATURE (4)..(5): polyethylene glycol (PEG) backbone modification between residues 4 and 5

SEQ ID NO: 50 - protein, 6 aa, Homo sapiens
TVLTAA

SEQ ID NO: 51 - protein, 6 aa, Homo sapiens
TLVTLL

SEQ ID NO: 52 - protein, 6 aa, Homo sapiens
TAITII

SEQ ID NO: 53 - protein, 6 aa, Homo sapiens
SVLTVV

SEQ ID NO: 54 - protein, 6 aa, Homo sapiens
SVLSVV

SEQ ID NO: 55 - protein, 6 aa, Homo sapiens
SVLTVV

SEQ ID NO: 56 - protein, 6 aa, Homo sapiens
TVLTVV

SEQ ID NO: 57 - protein, 11 aa, Homo sapiens
VVVVVTVVTVV

SEQ ID NO: 58 - protein, 11 aa, Homo sapiens
AAAAATVATVV

SEQ ID NO: 59 - protein, 11 aa, Homo sapiens
IIIIITVITVV

SEQ ID NO: 60 - protein, 11 aa, Homo sapiens
LLLLLTLLTLL

SEQ ID NO: 61 - protein, 11 aa, Homo sapiens
LLLLLSVLTVV

SEQ ID NO: 62 - protein, 11 aa, Homo sapiens
LLLLLSVLSVV

SEQ ID NO: 63 - protein, 11 aa, Homo sapiens
LLLLLTVLTVV

SEQ ID NO: 64 - protein, 1255 aa, Homo sapiens; identical to SEQ ID NO: 45

(19) United States
(12) Patent Application Publication
(10) Pub. No.: US 2023/0002446 A1
(43) Pub. Date: Jan. 5, 2023

(54) ANTI-INFLAMMATORY AGENTS

(71) Applicant: The University of Sydney, New South Wales (AU)

(72) Inventors: Richard J. PAYNE, New South Wales (AU); Scott N. BYRNE, New South Wales (AU); David M. McDONALD, New South Wales (AU); Anneliese S. ASHHURST, New South Wales (AU)

(21) Appl. No.: 17/616,809

(22) PCT Filed: Jun. 5, 2020
(86) PCT No.: PCT/AU2020/050572

§ 371 Date: Dec. 6, 2021

(30) Foreign Application Priority Data
Jun. 6, 2019 (AU) 2019901968

(51) Int. Cl. C07K 7/06; A61P 17/06

(52) U.S. Cl. CPC C07K 7/06; A61P 17/06

(57) ABSTRACT

The invention relates to anti-inflammatory peptides, to pharmaceutical compositions comprising same and to uses thereof for treatment of inflammation including, but not limited to inflammation associated with immune activation.

### RELATED APPLICATION

This application is a U.S. National Phase application, filed under 35 U.S.C. § 371, of International Application No. PCT/AU2020/050572, filed on Jun. 5, 2020, which claims priority to, and the benefit of, Australian provisional application AU 2019901968, filed on Jun. 6, 2019, the entire contents of each of which are hereby incorporated by reference.

### SEQUENCE LISTING

The instant application contains a Sequence Listing which has been submitted in ASCII format via EFS-Web and is hereby incorporated by reference in its entirety. Said ASCII copy, created on Jun. 23, 2022, is named “FRPA-026_N01US_SeqList.txt” and is about 91,619 bytes in size.

### FIELD OF THE INVENTION

The invention relates to anti-inflammatory peptides, to pharmaceutical compositions comprising same and to uses thereof for treatment of inflammation including but not limited to inflammation associated with immune response.

### BACKGROUND OF THE INVENTION

Reference to any prior art in the specification is not an acknowledgment or suggestion that this prior art forms part of the common general knowledge in any jurisdiction or that this prior art could reasonably be expected to be understood, regarded as relevant, and/or combined with other pieces of prior art by a skilled person in the art.

Inflammatory processes may be associated with, or caused by aberrant activation of the immune system, resulting in self-perpetuating cycles of inflammation and immune activation and subsequent immune responses that can be highly debilitating.

These processes may be causative of acute and chronic inflammation, including inflammation observed in cutaneous, musculoskeletal, enteric or pulmonary disorders or diseases.

Processes associated with, or caused by aberrant immune cell responses including aberrant macrophage or dendritic cell activation are of particular concern. One particular example is psoriasis which is a particularly problematic incurable condition estimated to affect 1-4% of the Western population that is characterized by epidermal hyperplasia and excessive inflammatory infiltrate in the skin.

Other inflammatory diseases or disorders may be idiopathic with suspected immune involvement, examples of cutaneous diseases including contact and atopic dermatitis, Polymorphic Light Eruption and Vitiligo.

Some of the current treatments have significant immune suppressive side effects or other adverse events.

There is a need for therapies that provide for prevention or treatment of inflammatory responses.

There is a need for therapies that provide for prevention or treatment of inflammatory responses associated with or caused by aberrant activation of the immune system.

### SUMMARY OF THE INVENTION

The invention seeks to address one or more of the above mentioned limitations or needs. Accordingly, in a first aspect, the invention provides an anti-inflammatory peptide for preventing or treating inflammation in an individual requiring said prevention or treatment, the peptide comprising, consisting of or consisting essentially of:
the amino acid sequence TVLTVV (SEQ ID NO:1), or the amino acid sequence LLLLLTVLTVV (SEQ ID NO:2) or functional variants thereof;

wherein the peptide consists of less than 21 amino acid residues, preferably from 6 to 20 amino acid residues.

In a preferred embodiment, the invention provides a peptide consisting of the amino acid sequence of SEQ ID NO:1.

In a further preferred embodiment, the invention provides a peptide consisting of the amino acid sequence of SEQ ID NO:2.

In alternative embodiments of the invention, the anti-inflammatory peptide comprises or consists of or consists essentially of the amino acid sequence of SEQ ID NO: 30, or functional variants or fragments thereof. More preferably, the peptide consists of the amino acid sequence of SEQ ID NO: 30.

In a further aspect of the invention, there is provided a polypeptide comprising:
a peptide comprising or consisting of the amino acid sequence of SEQ ID NO: 1 or SEQ ID NO: 2; and
one or more further amino acids adjacent to the sequence of SEQ ID NO: 1 and 2, provided that the one or more further amino acids do not define the sequence MTPGTQSPFF at a position N terminal adjacent to the sequence of SEQ ID NO: 1 or 2; or the sequence TGSGHASSTP at a position C terminal adjacent to the sequence of SEQ ID NO: 1 or 2,

wherein the polypeptide comprises at least 20 amino acid residues, preferably from 20 to 1000 residues.

In another aspect of the invention, there is provided an anti-inflammatory peptide comprising:
a peptide as defined herein, preferably a peptide comprising a sequence of any one of SEQ ID NOs: 1, 2, (preferably SEQ ID NO: 3 or 30), or functional variants or fragments thereof;
optionally one or more modifications selected from:
   a modification for facilitating the solubilisation of the peptide in a biological fluid, tissue or formulation,
   a moiety for facilitating the entry of the peptide into a cell, and
   a modification or moiety for enabling detection of the peptide.

In embodiments of this aspect of the invention, the peptide preferably comprises an amino acid sequence that is at least 90%, at least 95% or at least 98% identical to the sequence of SEQ ID NO:1, SEQ ID NO:2, SEQ ID NO: 3 or SEQ ID NO: 30.

The invention provides a peptide consisting of the sequence according to any one of SEQ ID NOs: 1 to 65 or functional variants or fragments thereof. Functional variants or fragments generally exhibit at least 70%, 71%, 72%, 73%, 74%, 75%, 76%, 77%, 78%, 79%, 80%, 70%, 81%, 82%, 83%, 84%, 85%, 86%, 87%, 88%, 89%, 90%, 91%, 92%, 93%, 94%, 95%, 96%, 97%, 98%, or at least 99% amino acid identity and retain their function in treating or preventing an inflammatory response as described herein including in the Examples.

In any embodiment, the modification for facilitating the solubilisation of the peptide is in the form of an amino acid residue (or series thereof), polymer or other functional moiety that promotes the solubilisation of the peptide. In certain embodiments, the modification for facilitating the solubilisation of the peptide comprises polyethylene glycol (PEG) of varying lengths. For instance, PEG may comprise at least 1, 2, 3, 4, 5, 6, 7, 8, 9, 10, 11, 12, 13, 14, 15 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or more ethylene glycol units. Alternatively, PEG may comprise no ethylene glycol units.

In further embodiments, the modification for facilitating the solubilisation of the peptide comprises one or more charged amino acid residues, preferably one or more cationic amino acid residues, more preferably one or more of lysine (K), arginine (R), or histidine (H).

It will be appreciated that the modification for facilitating the solubilisation of the peptide can be located at the N or C terminal regions of the peptide. Preferably, the modification is located at the N terminal region of the peptide.

One or more modifications for improving the solubilisation of the peptide can be used, for example, a combination of a polymer and cationic amino acid residues.

In any embodiment, the moiety for detecting the peptide is any label known in the art for facilitating detection of the peptide in vivo or in vitro. The label may be a fluorescent label such as GFP, YFP, BFP, CFP, Y-FAST, AF594, mCherry or dsRed. The label may be biotin. Alternatively, the label may be a metal label such as (Gd)Dota.

In yet another embodiment, the peptides described herein may contain synthetic variants of amino acid residues. In certain embodiments, the synthetic amino acid variant is photoleucine.

In any embodiment, a peptide described herein may comprise a cell penetrating peptide (CPP) conjugated to the N- or C-terminal region to facilitate or assist the entry of any peptide herein into a cell. Non-limiting examples of cell penetrating peptides are well known in the prior art and are further described herein. Non-limiting examples include Tat and peptides based on Tat.

In particularly preferred embodiments of the invention, the anti-inflammatory peptide comprises or consists of or consists essentially of the peptide as defined in SEQ ID NO: 12: KKKK-PEG-MTPGTQSPFFLLLLLTVLTVV. More preferably, the peptide is as defined in SEQ ID NO: 12.

In certain embodiments, a peptide of the invention may further be conjugated to any anti-inflammatory peptide known in the art.

In any embodiment of the invention, the peptides described herein comprise a linker comprising varying lengths of glycine or serine repeats. The peptide of the invention and the linker may be directly or indirectly linked. Preferably, the linker is located at the N-terminus of the peptide, although it will be appreciated that the linker may also be located at the C-terminus of the peptide. In an embodiment, the linker comprises the sequence GGG and a peptide according to the invention comprises or consists of KKKK-GGG-MTPGTQSPFFLLLLLTVLTVV (SEQ ID NO: 22) or functional variants thereof (including KKKK-GGG-TPGTQSPFFLLLLLTVLTVV (SEQ ID NO: 23).

The anti-inflammatory peptide may comprise, consist or consist essentially of any peptide as described in any one of Tables A-E herein.

In any embodiment there is provided a pharmaceutical composition comprising any one of the peptides described herein (including any one of the peptides of SEQ ID NO: 1 to 66), or functional variants thereof, and a pharmaceutically acceptable carrier, diluent or excipient, or a pharmaceutically acceptable salt thereof.

In certain embodiments, the pharmaceutical composition comprises a peptide comprising or consisting of the sequence of SEQ ID NOs: 1, 2, 3, 12, or 30 and a pharmaceutically acceptable carrier, diluent or excipient, or a pharmaceutically acceptable salt thereof.

In yet another embodiment, any peptide or composition comprising a pharmaceutically effective amount of any one of the peptides described herein or functional variants thereof, or a pharmaceutically acceptable salt thereof, does not comprise granulocyte-macrophage colony-stimulating factor (GM-CSF).

The present invention also provides a composition comprising any peptide described herein and a further active agent. The further active agent may be an anti-inflammatory agent, a non-steroidal anti-inflammatory drug (NSAID), a disease modifying antirheumatic drug (DMARD), or other active agent useful for the treatment of the inflammatory disorder requiring treatment (e.g., agent for treating psoriasis, dermatitis, rheumatoid arthritis as the case may be).

In certain embodiments, the anti-inflammatory agent is a corticosteroid. Non-limiting examples of corticosteroids suitable for use in the invention include aristocort, decadron, mometasone, cotolone, triamcinolone, cortisone, prednisone, methylprednisolone and betamethasone dipropionate.

Alternatively, the further anti-inflammatory agent is an antigen binding protein for inhibiting the activity of a pro-inflammatory molecule. Preferably, the antigen binding protein is an antibody or antigen binding fragment thereof. Typically, the antigen binding protein is an antibody, for example, a monoclonal antibody.

In certain embodiments, the antigen binding protein (e.g., monoclonal antibody) for inhibiting the activity of a pro-inflammatory molecule, binds to an anti-inflammatory molecule, thereby inhibiting the activity of the molecule in promoting an inflammatory response. The antigen binding protein may bind to a receptor of the pro-inflammatory molecule, thereby inhibiting the activity of the pro-inflammatory molecule. Non-limiting examples of suitable antigen binding proteins or monoclonal antibodies for use in accordance with the invention include those that are capable of inhibiting the activity of a pro-inflammatory molecule including interleukin-17 (IL-17), interleukin-12 (IL-12), interleukin-6 (IL-6), interleukin-22 (IL-22), interleukin-23 (IL-23), interleukin-36 (IL-36), and tumour necrosis factor-alpha (TNFα). Examples of such antigen binding proteins or monoclonal antibodies include brodalumab, ixekizumab or secukinumab (for inhibiting IL-17), tocilizumab or siltuximab (for inhibiting IL-6), guselkumab or tildrakizumab or mirikizumab or brazikumab or risankizumab or ustekinumab (for inhibiting IL-23), fezakinumab (for inhibiting IL-22), etanercept, infliximab, adalimumab, certolizumab, certolizumab pegol or golimumab (for inhibiting the activity of TNFα).

In embodiments where the further active agent is a DMARD, the DMARD may be thalidomide, lenalidomide, methotrexate.

In still further embodiments, the further active agent may be any agent known to be useful for treating an inflammatory disorder, non-limiting examples which include: calcipotriol, Anakinra, inflammasome inhibitors, Baricitinib, Abrocitinib, Upadacitinib (JAK inhibitors), tofacitinib, ruxolitinib, antihistamines, topical tacrolimus (a calcineurin inhibitor) and crisaborole (a phosphodiesterase 4 (PDE4) inhibitor).

A composition comprising any peptide described herein and a further anti-inflammatory agent may provide an anti-inflammatory effect that is greater than the effect of the peptide alone. For example, the effect of any peptide described herein and a corticosteroid is synergistic, compared to the effect of the individual components alone. As such, the present invention also includes synergistic compositions comprising a peptide as described herein, and a further anti-inflammatory agent, wherein the further anti-inflammatory agent is preferably a corticosteroid, DMARD or antigen binding protein for binding to inhibiting the activity of a pro-inflammatory molecule.

In any embodiment, the composition comprising any peptide described herein and the further anti-inflammatory agent may be formulated for administration in the form of a tablet, syrup or liquid, inhaler or nasal spray, injection (including formulations for intradermal or subcutaneous injection, mesotherapy/microneedling), patch or as a cream, ointment, spray, lotion or gel.

In another embodiment, there is provided nucleic acids or fragments thereof that encode the peptides defined herein comprising a sequence of DNA or RNA, including one having an open reading frame that encodes the therapeutic polypeptide and is capable, under appropriate conditions, of being expressed as one of the therapeutic peptides of the instant invention. Alternatively, the invention provides for vectors or plasmids comprising the above nucleic acids or fragments thereof.

In a further aspect of the invention, there is provided a method for preventing or treating inflammation or an inflammatory disorder in an individual comprising administering a peptide or a pharmaceutically effective amount of a composition described herein to an individual in need thereof, thereby preventing or treating inflammation in the individual.

The inflammation may be located in or on tissue selected from skin or mucosal tissue, musculoskeletal, pulmonary or enteric tissue. In certain preferred embodiments, the methods of the invention are for treating inflammation in dermal tissue.

The inflammation may be a symptom of a disease or condition selected from the group consisting of an inflammatory skin disease or disorder, a musculoskeletal disease or disorder, a pulmonary disease or disorder, or an enteric disease or disorder.

The inflammatory disorders and related conditions which may be treated or prevented by use of the peptides or compositions described herein include, but are not limited to rheumatoid arthritis, bronchitis, contact dermatitis, atopic dermatitis, psoriasis, seborrheic dermatitis, eczema, allergic dermatitis, polymorphous light eruptions, inflammatory dermatoses, folliculitis, alopecia, vitiligo, poison ivy, insect bites, acne inflammation, irritation induced by extrinsic factors including, but not limited to, chemicals, trauma, pollutants (such as cigarette smoke) and sun exposure, secondary conditions resulting from inflammation including but not limited to xerosis, hyperkeratosis, pruritus, post-inflammatory hyperpigmentation, scarring, lung inflammatory conditions (including influenza), lupus, rheumatoid arthritis, multiple sclerosis, sarcoidosis and scleroderma, hypersensitivity pneumonitis, wound healing and inflammatory bowel disease including Colitis and Crohn's disease or inflammation resulting from transplant (graft versus host disease, GVHD).

In preferred embodiments, where the inflammatory disorders and related conditions is an inflammatory skin disorder, the disorder may be contact dermatitis, atopic dermatitis, allergic dermatitis, or psoriasis. In such embodiments, the administration of a peptide or composition described herein minimises one or more symptoms selected from the group consisting of erythema, skin thickness, scaling and inflammatory oedema.

In another embodiment, the inflammation to be treated is associated with TNF, IL-6 or IL-17 or IL-12/23 production. Thus, the administration of a peptide or composition described herein may minimise the release of inflammatory cytokines, preferably TNF, IL-6, IL-17 and IL-12/23.

In another embodiment, the administration of a peptide or composition described herein minimises the activation of lymphocytes, neutrophils or mast cells or antigen presenting cells, preferably macrophages or dendritic cells at the site of inflammation.

In another embodiment, there is provided a method of regulating an immune response in an individual comprising administering a peptide, or a pharmaceutically effective amount of a composition described herein to an individual in need thereof, thereby regulating an immune response in the individual. Regulation of an immune response will be understood to be an effect observed in any of the examples demonstrated herein in response to the peptides or compositions of the invention, or as understood by a person skilled in the art.

In an embodiment, the immune response is an adaptive or innate immune response. Where the immune response is an innate immune response, the innate immune response is associated with activation of antigen presenting cells, preferably macrophages or dendritic cells.

In any embodiment, the methods of the invention may additionally comprise administration of a further active agent including an anti-inflammatory agent, a non-steroidal anti-inflammatory drug (NSAID), a disease modifying antirheumatic drug (DMARD), or other active agent useful for the treatment of the inflammatory disorder requiring treatment (e.g., agent for treating psoriasis, dermatitis, rheumatoid arthritis as the case may be) or administration of a further treatment (such as the use of phototherapy).

In certain embodiments, the methods comprise administration of a further anti-inflammatory agent in the form of a corticosteroid. Non-limiting examples of corticosteroids suitable for use in methods of the invention include aristocort, decadron, mometasone, cotolone, triamcinolone, cortisone, prednisone, methylprednisolone and betamethasone dipropionate.

Alternatively, the methods may comprise administration of an anti-inflammatory agent that is antigen binding protein for inhibiting the activity of a pro-inflammatory molecule. Preferably, the antigen binding protein is an antibody or antigen binding fragment thereof. Typically, the antigen binding protein is an antibody, for example, a monoclonal antibody. The antigen binding protein (e.g., monoclonal antibody) may be any antigen binding proteins or monoclonal antibodies capable of inhibiting the activity of a pro-inflammatory molecule including interleukin-17 (IL-17), interleukin-12 (IL-12), interleukin-6 (IL-6), interleukin-22 (IL-22), interleukin-23 (IL-23), interleukin-36 (IL-36), and tumour necrosis factor-alpha (TNFα). Examples of such antigen binding proteins or monoclonal antibodies include brodalumab, ixekizumab or secukinumab (for inhibiting IL-17), tocilizumab or siltuximab (for inhibiting IL-6), guselkumab or tildrakizumab or mirikizumab or brazikumab or risankizumab or ustekinumab (for inhibiting IL-23), fezakinumab (for inhibiting IL-22), etanercept, infliximab, adalimumab, certolizumab, certolizumab pegol or golimumab (for inhibiting the activity of TNFα).

In embodiments where the further active agent is a DMARD, the DMARD may be thalidomide, lenalidomide, methotrexate.

In still further embodiments, the further active agent may be any agent known to be useful for treating an inflammatory disorder, non-limiting examples which include: calcipotriol, Anakinra, inflammasome inhibitors, Baricitinib, Abrocitinib, Upadacitinib (JAK inhibitors), tofacitinib, ruxolitinib, antihistamines, topical tacrolimus (a calcineurin inhibitor) and crisaborole (a phosphodiesterase 4 (PDE4) inhibitor).

In another embodiment there is provided a use of a peptide described herein in the manufacture of a medicament for:
prevention or treatment of inflammation, preferably inflammation associated with aberrant immune activation, optionally contact dermatitis, atopic dermatitis, allergic dermatitis or psoriasis;
regulating an immune response, preferably an innate immune response;
reducing levels of TCRαβ⁺ T-cells;
reducing levels of TCRγδ+ T cells;
increasing regulatory cells, preferably CD4⁺ T regulatory cells; or
inhibiting the activation of neutrophils, mast cells or antigen presenting cells, preferably macrophages or dendritic cells at the site of inflammation.

In a further embodiment, the medicament may further comprise an anti-inflammatory, preferably a corticosteroid for preventing or treating an inflammatory response. In yet another embodiment, the medicament may further comprise an antigen-binding protein for binding to a pro-inflammatory molecule, preferably selected from the group consisting of interleukin-17 (IL-17), interleukin-12 (IL-12), interleukin-6 (IL-6), interleukin-23 (IL-23), interleukin-36 (IL-36) and tumour necrosis factor (TNF).

In yet another embodiment, there is provided:
use of a peptide described herein in the manufacture of a first medicament; and
use of a corticosteroid for preventing or treating an inflammatory response in the manufacture of a second medicament; or
use of a an antigen-binding site for inhibiting the activity of a pro-inflammatory molecule, preferably selected from the group consisting of interleukin-6 (IL-6), interleukin-17 (IL-17), interleukin-12 (IL-12), interleukin-23 (IL-23), interleukin-36 (IL-36) and tumour necrosis factor (TNF) in the manufacture of a second medicament,

for:
prevention or treatment of inflammation, preferably inflammation associated with aberrant immune activation, more preferably contact dermatitis, atopic dermatitis, allergic dermatitis or psoriasis;
regulating an immune response, preferably an innate immune response;
reducing levels of TCRαβ⁺ T-cells;
reducing levels of TCRγδ+ T cells;
increasing regulatory cells, preferably CD4⁺ T regulatory cells; or
inhibiting the activation of neutrophil, mast cells or antigen presenting cells, preferably macrophages or dendritic cells at the site of inflammation.

In another embodiment there is provided a peptide or a pharmaceutically effective amount of a composition as described above for use in the prevention or treatment of inflammation, preferably inflammation associated with aberrant immune activation.

In any embodiment, the peptides or compositions described herein are suitable for administration via intradermal, intra-articular, intramuscular, intraperitoneal, intravenous, subcutaneous, intranasal, epidural, oral, sublingual, intracerebral, intra-lymph node, intratracheal, intravaginal, transdermal, rectally, by inhalation, or topically, particularly to the ears, nose, eyes, or skin. In a preferred embodiment, the peptides or compositions described herein are administered topically, intranasally or via inhalation.

As used herein, except where the context requires otherwise, the term “comprise” and variations of the term, such as “comprising”, “comprises” and “comprised”, are not intended to exclude further additives, components, integers or steps.

Further aspects of the present invention and further embodiments of the aspects described in the preceding paragraphs will become apparent from the following description, given by way of example and with reference to the accompanying drawings.

### BRIEF DESCRIPTION OF THE DRAWINGS

FIG. 1—Peptide administration leads to accelerated skin tumour growth. Mice received sub-cutaneous injections of mixtures containing 5 nmol peptide RP23 (SEQ ID NO:12) (solid line), PBS or liposome controls (dotted line) 21 and 7 days prior to transfer of UV13-1 tumour cells. If peptide RP23 was included in the vaccine formulation, tumours grew faster (A) and mice succumbed to their tumour earlier (B). Difference in survival was analysed statistically using a log-rank test.

FIG. 2—Peptide administration reduces inflammation in a mouse model of contact dermatitis and reduces myeloid inflammatory infiltrate. (A) Experimental outline of DNFB-induced contact dermatitis. (B) Ear thickness of peptide RP23 (SEQ ID NO:12) treated mice, repeated 3 times. (C) Inflammatory infiltrate in the ear was characterised by flow cytometry at 72 hours post challenge with DNFB (D15), and demonstrated alterations to monocyte/macrophage populations (CD11b⁺). Data are presented as mean±S.E.M. Statistical significance was determined (B) by multiple t tests (C) by ANOVA with Dunnett's multiple comparison test, *p<0.05, **p<0.01, ***p<0.001.

FIG. 3—The peptide effect is dependent on the peptide sequence and dose. (A) PBS, scrambled peptide or peptide MUC1SP (MTPGTQSPFFLLLLLTVLTVV; SEQ ID NO: 3) was injected subcutaneously seven days prior to sensitising C57BL/6 mice (n=5) with DNFB on the abdomen. Ear inflammation after DNFB challenge on the ear was determined, demonstrating that the effect was specific to the SEQ ID NO: 3 sequence. (B) A comparison of the immunosuppressive effect of peptide RP23 (SEQ ID NO:12) and MUC1SP (SEQ ID NO: 3). (C) SEQ ID NO:12 suppressed immunity in a dose dependent manner. (D) Addition of GM-CSF to the SEQ ID NO:12 injection abrogated the immune suppression. Data are the means±S.E.M.

FIG. 4—Peptide administration reduces local disease severity in a mouse model of psoriasis. (A) Experimental outline (B) Clinical scores of mice receiving PBS or peptide RP23 (SEQ ID NO:12) injection, compared to daily (0-4 days) topical application of glucocorticoid on the back and ear. (C) Measures of disease local to the peptide RP23 injection site. Redness was measured using an unbiased erythema meter and skin thickness by manual callipers. (D) Spleen weight at day 5 as a measure of systemic immune suppression. Ear thickness measurements indicated peptide RP23 did not suppress disease at a distal site. Data are presented as mean±S.E.M. Statistical significance was determined by ANOVA with Dunnett's multiple comparison test, *p<0.05, **p<0.01, ***p<0.001, ****p<0.0001.

FIG. 5—Peptide administration reduces the activation of pro-inflammatory DCs and macrophages. (A) C57BL/6 murine splenocytes were stimulated with LPS±peptide RP23 (SEQ ID NO:12) and expression of activation markers on DCs was assessed by flow cytometry. (B) Bone marrow derived murine macrophages were incubated with peptide RP23 (30 mins) and stimulated with LPS (overnight). Positive controls were stimulated with LPS only. Negative controls were unstimulated. Inflammatory cytokine production was assessed by ELISA. (C) CD14+ cells were isolated from human blood and differentiated into macrophages by culturing with human serum. Cells were incubated with peptide RP23 (30 mins), then stimulated overnight with LPS (left panel) or imiquimod (right panel). Inflammatory cytokine (IL-6 or IL-12/23) production was assessed by ELISA. Representative of 6 donors. Data are presented as mean±S.E.M. Statistical significance was determined by ANOVA with Dunnett's multiple comparison test, *p<0.05, **p<0.01, ***p<0.001.

FIG. 6—Peptide administration is not cytostatic or cytotoxic to murine and human macrophages/monocytes. Cytostatic/cytotoxic effect of peptide RP23 (SEQ ID NO:12) was assessed after overnight incubation (A) by WST-1 assay in RAW and THP-1 cell lines (data are the means of 2 replicates) (B) by LDH assay in primary murine BMDMs or human MDMs (data are mean±S.E.M).

FIG. 7—Peptide administration reduces spontaneous myeloid cell activation in human skin. (A) Peptide RP23 (SEQ ID NO: 12) was injected intradermally into healthy human skin grafts. Myeloid cell activation was assessed after overnight culture. (B) The effect of peptide RP23 on the spontaneous upregulation of activation markers on myeloid cell subsets in the skin. Data are representative of 6 donors.

FIG. 8—Effects of peptide variants on the inflammatory response. Mice received one injection of PBS or one of the following peptides (10 nmol) subcutaneously at the abdomen (A) peptide RP23 (SEQ ID NO: 12), peptide AF594-RP23 (SEQ ID NO: 21), peptide K4-G3-MUC1SP (SEQ ID NO:22) or MUC1SP C fragment (SEQ ID NO: 2). Five days later, mice were challenged with DNFB at a distal site, on the ears. The thickness of the ears was measured over 72 hours.

FIG. 9—Effects of further peptide variants, without N-terminal methionine, on the inflammatory response in a model of contact dermatitis. Mice received one injection of PBS, RP23 (SEQ ID NO:12) (10 nmol) or one of the peptide variants indicated in the table including SEQ ID NO: 32 and 35 as described herein (equivalent molar amount of RP23), subcutaneously at the abdomen. Photoleucine replaces leucine where indicated, these molecules used for photo-crosslinking studies. Seven days later, abdominal skin was sensitised with a contact irritant (DNFB) and five days later, mice were challenged with DNFB at a distal site, on the ears. (A) Specific increase in ear thickness over 72 hours, data are the means+/−SEM (n=6). Statistical significance was determined by ANOVA with Dunnett's multiple comparison test to PBS only control, **p<0.01. (B) Specific increase in ear thickness at 24 hours post-challenge. Data are the means+/−SEM (n=6). Statistical significance was determined by t-test to relevant control, as indicated, *p<0.05, **p<0.01, ns=not significant.

FIG. 10. Therapeutic synergy of RP23 and topical steroid in the murine imiquimod-induced psoriasis model. Aldara cream (5% imiquimod, 57.5 mgs) was applied daily (days 0-4) to the backs of C57BL/6 female mice to induce psoriasis. Clinical scores (PASI), modified psoriasis area and severity index) of mice receiving (A) PBS or RP23 injection at day −1, with or without topical application of glucocorticoid (0.05% betamethasone dipropionate, “BD” 30 mgs) on the back at day 0 (B) PBS or RP23 injection at day 1 and topical application of glucocorticoid at day 1, in comparison to PBS injection only at day −1. Data are the means+/−SEM (n=5). Statistical significance was determined by ANOVA with Dunnett's multiple comparison test to PBS only control, *p<0.05, **p<0.01, ***p<0.001, ****p<0.0001.

FIG. 11. RP23 treatment reduces TCRαβ⁺ T-cells in the back skin and increases CD4⁺ T-regulatory cells in the lymph nodes of mice in an imiquimod-induced psoriasis model. C57BL/6 female mice received PBS or RP23 injection at day −1, then Aldara cream (5% imiquimod, 57.5 mgs) was applied daily (days 0-4) to the backs to induce psoriasis. At day 15, skin and inguinal lymph nodes were analysed by flow cytometry to quantitate (A) T-cell subsets as a proportion of total leukocytes (CD45⁺) in the psoriatic back skin, (B) number of CD3⁺CD4⁺ TCRαβ T-cells in the skin draining lymph nodes (inguinal), with transcription factor staining, to identify cells with a regulatory phenotype (FoxP3⁺). Data are the means+/−SEM (n=4-5). Statistical significance was determined by multiple t-tests, *p<0.05.

FIG. 12. RP23 formulated for topical application can penetrate human skin. RP23^{AF594} in a stock emulsion was applied topically to full thickness human skin explants and cultured for 24 hours. Frozen sections were analysed by fluorescence microscopy. RP23^{AF594} (association with DAPI+ cells indicated by arrows) was detectable in both the epidermis (E) & below the basement membrane (line) in the dermis (D).

FIG. 13. RP23 treatment reduces inflammation in a murine model of atopic dermatitis. Ears and shaved backs of C57BL/6 female mice were sensitised (day 0) and challenged (day 7, 10, 14, 17, 19, 21, 23, 25 and 27) with topical application of the irritant oxazolone or vehicle only. Injection of mice with RP23 at day −1, or both day −7 and day −1, is compared to injection with PBS only (day −7 and day −1) or topical application of steroid (0.01% betamethasone dipropionate, 30 mgs) applied on the back at the time of every oxazolone application. Ear thickness was measured with manual callipers. Data are the means+/−SEM (n=5).

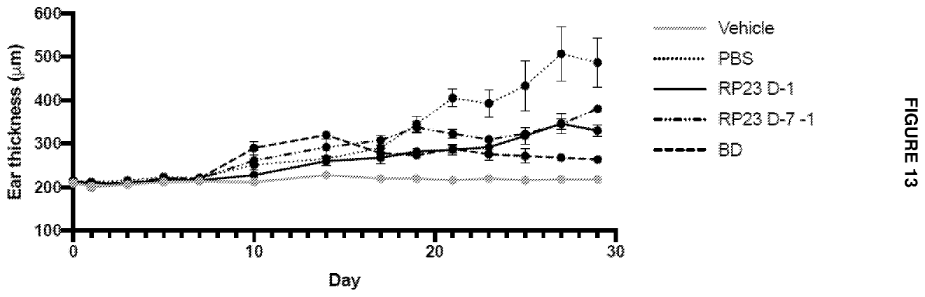

FIG. 14. RP23 interacts with innate immune cells in the skin and lungs. AF594-RP23⁺ cells were identified by flow cytometry 24 hours after administration to C57BL/6 mice. (A) RP23 was injected subcutaneously at the lower back. RP23⁺ leukocytes (CD45⁺) in the back skin were primarily neutrophils (CD11b⁺ Ly6G⁺) or CD11b⁺ myeloid cell subsets. (B) RP23 was delivered to the lungs by intranasal instillation. The percentage distribution of total RP23⁺ leukocytes amongst immune cell subsets in the lungs is shown. Data are the means+/−SEM (n=4).

FIG. 15. Mucosal delivery of RP23 to the lungs decreases activation of antigen presenting cells in the lungs and bronchoalveolar space. (A) C57BL/6 mice (n=5) received either PBS or RP23 by intra-nasal instillation or (B) C57BL/6 mice (n=4) received either PBS, scramble RP23 peptide, or RP23 by intra-nasal instillation, then 24 hours later, Pam₂Cys-SK₄-PEG(OH) intra-nasally to induce an inflammatory state. 24 hours later, cells from the bronchoalveolar lavage and perfused lungs were collected. The median fluorescence intensity of MHCII on CD11c⁺ dendritic cells was determined by flow cytometry. Data are the means+/−SEM. Statistical significance was determined by (A) t-test, ***p<0.001 and (B) ANOVA with Dunnett's multiple comparison test to PBS control, *p<0.05.

### DETAILED DESCRIPTION OF THE EMBODIMENTS

The inventors have herein identified a class of peptides that are useful for the suppression, prevention or treatment of inflammation. In particular, the inventors have established that the peptides characterised herein have the ability to suppress, prevent or treat an inappropriate inflammatory response. In an embodiment, the peptides can be used to suppress an inflammatory response that is associated with immune system activation. In another embodiment, the peptides described herein can be used to suppress an inflammatory response in the absence of an accompanying immune system activation.

The peptides as described herein have been shown to have utility in a number of models that are commonly used to assess activation of the inflammatory response, including dermatitis and psoriasis. These models are well characterised in the art to comprise episodes of acute inflammation and/or chronic inflammation. The inventors therefore recognise the application of the defined peptides for a wide range of diseases or conditions which are accompanied by inflammation, of which those disclosed are just some examples.

The peptides described herein have particular utility in the suppression of erythema which is a key feature of the inflammatory process. In one aspect, this is achieved through the suppression of monocytic/macrophage or other phagocytic cell infiltration into an effected area. In particular, the peptides are capable of reducing the secretion of inflammatory chemokines or cytokines and the expression of surface activation markers of pro-inflammatory immune cells. Non-limiting examples include CD86, MHCII and levels of IL-6, IL-12/IL-23, TNF-alpha and MCP-1. The inventors also establish the surprising finding that the effect of suppression of the inflammatory response is in the absence of any cytostatic or cytotoxic effects to the cells. The peptides described herein surprisingly can reduce activation of antigen presenting cells, reduce spontaneous activation of dermal dendritic cells, reduce levels of TCRαβ⁺ T cells and increase CD4⁺ T regulatory cells. These data point to a potential role for the peptides in inhibiting inflammation in a wide range of inflammatory conditions, including any inflammatory condition mediated by TNF-α and other pro-inflammatory cytokines or any inflammatory condition suppressed by the induction of regulatory cells like CD4⁺ T regulatory cells.

The data obtained by the inventors suggests that the peptides of the invention suppress pro-inflammatory responses by innate immune cells that are involved in the stimulation of inflammation under many different stimuli, including autoinflammation, tissue damage, cancer or infection. Using models that are either characteristically Th1-type inflammation, or IL-17/Th17 driven, the inventors show that the peptides of the invention may moderate (reduce) stimulation of Th1/Th17 type responses, and/or promote a regulatory response (FoxP3+CD4+ T-cells). These data therefore indicate that the peptides of the invention are likely to be suitable in the treatment of inflammatory disorders including indications such as psoriasis, dermatitis, rheumatoid arthritis, IBD, transplant rejection/tolerance (graft versus host disease, GVHD).

The peptides characterised herein therefore have utility in suppressing, treating or preventing an inflammatory response that is characterised by one or more of:

1. erythema;

2. monocytic/macrophage infiltration;

3. increased levels of cytokines and/or chemokines;

4. activation of cell receptors/markers on inflammatory cells;

5. activation of dendritic cells; and/or

6. activation of lymphocytes.

The inventors also establish that the peptides described have utility in reducing local inflammation in a manner that avoids unwanted immune system suppression. This is important because most clinically prescribed immunosuppressant drugs cause systemic immune suppression. The peptides described herein therefore provide for an advantage not previously recognised.

### Inflammatory Responses

The peptides described herein have utility in treating or suppressing an acute or chronic inflammatory response with or without an accompanying immune response. Alternately, the peptides described herein can suppress an inappropriate acute inflammatory response that is associated with an immune response which may be an innate response, adaptive immune response, or both.

The term suppression of an inappropriate inflammatory response or treating an inappropriate immune response means a reduction in one or more of the symptoms of inflammation including erythema (redness), edema (swelling), pain and pruritus which are characteristic of inflammatory conditions. This may also be understood to be a reduction in one or more pro-inflammatory cytokines and/or chemokines such as TNF-alpha, IL-6, CXCL1, CXCL2, IL-1beta, prostaglandin E2 and IL-23/IL-17 as well as other chemokines/cytokines known in the art.

In an embodiment, the peptides described herein have utility in treating or suppressing an acute inflammatory response with or without an accompanying immune response. In an embodiment, the peptides can suppress an acute inflammatory response that is associated with an innate immune response.

Acute inflammation is understood to be a short-term process occurring in response to tissue injury, and usually appears within minutes or hours. It is characterized by five cardinal signs: pain, redness, immobility (loss of function), swelling and heat. The acute inflammatory response involves the recruitment of blood leukocytes, activation of tissue-resident cells (mast cells, dendritic cells, fibroblasts, macrophages), and production of a series of mediators including IL-1 and TNF alpha. The results of this may include ultimate resolution of the inflammatory process, triggering events that lead to cell regeneration or wound healing, or progression of the inflammatory response, which may be characterised as chronic inflammation.

In an embodiment, the peptides described herein have utility in treating or suppressing a chronic inflammatory response with or without an accompanying immune response. In an embodiment, the peptides can suppress a chronic inflammatory response that is associated with an adaptive immune response.

Chronic inflammation is understood to be slow, long-term inflammation lasting for prolonged periods of several months to years. Chronic inflammation may be caused by a number of varying factors including: (1) failure to eliminate an agent causing acute inflammation such as infectious organisms including Mycobacterium tuberculosis, protozoa, fungi, and other parasites that can resist host defenses and remain in the tissue for an extended period; (2) exposure to a low level of a particular irritant or foreign materials that cannot be eliminated by enzymatic breakdown or phagocytosis in the body; (3) an autoimmune disorder in which the immune system is sensitized to the normal component of the body; (4) recurrent episodes of acute inflammation; or (5) inflammatory and biochemical inducers that cause oxidative stress and mitochondrial dysfunction.

It will be understood that a chronic inflammatory process can be diagnosed by one of many known tests in the art. These include (1) serum protein electrophoresis (SPE) which shows concomitant hypoalbuminemia and polyclonal increase in all gamma globulins (polyclonal gammopathy); (2) measurement of serum high-sensitivity C-reactive protein (hsCRP) and fibrinogen (normal serum levels for hsCRP is less than 0.55 mg/L in men and less than 1.0 mg/L in women, and normal levels of fibrinogen are 200 to 300 mg/dl); and detecting pro-inflammatory cytokines like tumour necrosis factor-alpha (TNF-alpha), interleukin-1 beta (IL-1 beta), interleukin-6 (IL-6), interleukin-8 (IL-8), interleukin-17 (IL-17) and interleukin 12/23 (IL-12/23).

### Adaptive and Innate Immune Responses

The peptides described herein have utility in suppressing inflammatory processes that are associated with adaptive or innate immune responses.

Adaptive or acquired immunity involves humoral (B lymphocytes) and cell-mediated (T lymphocytes) immune responses. Humoral immunity involves B cells and their products, including antibodies. Cell-mediated immunity involves the activation of phagocytes, antigen-specific CD4+ and CD8+T-lymphocytes, and the release of various cytokines in response to an antigen. Cell mediated immunity results in the activation of antigen-specific cytotoxic T cells that are able to induce apoptosis in cells displaying epitopes of foreign antigen on their surface, such as virus-infected cells or cells with intracellular bacteria. A cell-mediated immune response, particularly CD4+ helper responses, also involves activation of B cells. A cell mediated immune response also involves macrophages and innate lymphocytes (e.g. natural killer cells) for the destruction of pathogens via recognition and secretion of cytotoxic granules (for natural killer cells) and phagocytosis (for macrophages), as well as the stimulation of cells to secrete cytokines. Cell-mediated immunity is largely T-cell-driven, utilising helper (CD4+) and cytotoxic (CD8+) subtypes, and is involved in responses to viral infection, graft rejection, chronic inflammation and tumour immunity. Activated T-cells can take several days to initiate a cell-mediated attack after exposure to a novel antigen, but memory T-cells respond rapidly if primed by previous exposure. The peptides described herein have utility in the treatment of chronic inflammatory processes associated with an adaptive immune response as known in the art and as described herein.

In contrast, the innate immune system consists of barriers to infection (epithelia of skin, gastrointestinal, respiratory, genitourinary tracts), antimicrobial peptides and proteins, humoral components (i.e. complement and opsonins) and cellular components (i.e. mast cells, neutrophils, monocytes/macrophages, dendritic cells, and innate lymphoid cells). Innate immunity serves as the front line of host defense and plays an essential role in preventing infection while tolerating normal host flora. The innate immune system directs the subsequent development of adaptive immune responses.

It will be understood that the peptides described herein have utility in suppressing an inappropriate inflammatory response associated with an innate immune response within such barrier layers of the body including the skin, gastrointestinal, joints and respiratory tract.

A skilled person will also understand that the inappropriate inflammatory response may be associated with activation of both the adaptive and innate immune responses.

### GM-CSF

In an embodiment, the peptide, pharmaceutical composition comprising a pharmaceutically effective amount of a peptide described herein, or a pharmaceutically acceptable salt thereof does not comprise granulocyte-macrophage colony-stimulating factor (GM-CSF).

GM-CSF is a monomeric glycoprotein secreted by macrophages, T cells, mast cells, natural killer cells, endothelial cells and fibroblasts that functions as a cytokine. It is known to have a pro-inflammatory role primarily due to its role as a growth and differentiation factor for granulocyte, macrophage and monocyte and dendritic cell populations. Thus, agents that can block GM-CSF or its receptor have been used as anti-inflammatory therapies.

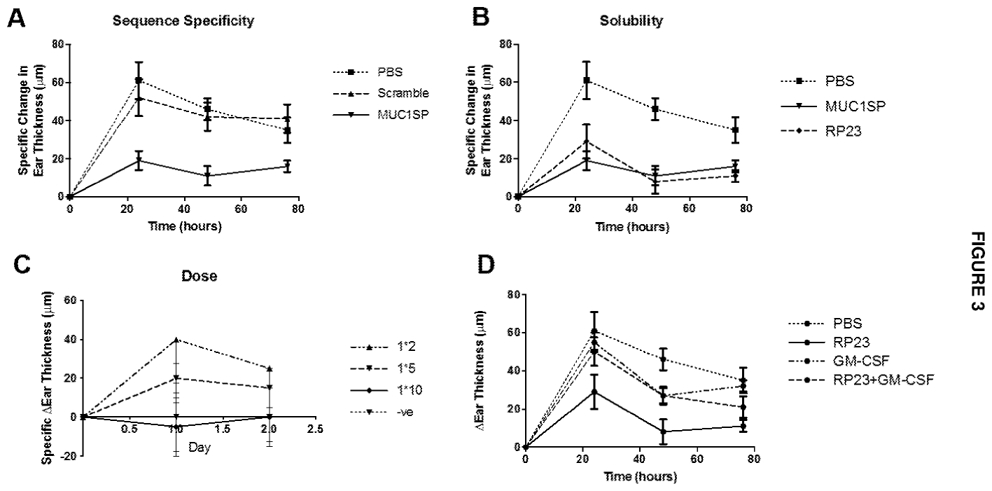

It is therefore envisaged that the peptides described herein with utility in the suppression, treatment or prevention of an inappropriate inflammatory response would not be used in combination with a growth factor, cytokine, chemokine or the like that promotes an inflammatory process as this may counteract the therapeutic effect of any of the peptides described herein. This is exemplified in the Examples herein whereby GM-CSF abrogates the suppressive effect of peptides of the invention (FIG. 3).

The determination of such growth factors, cytokines, chemokines or the like that may promote inflammation is well within the knowledge of a skilled person and may include GM-CSF, TNF-alpha, IL-6, IL-12, IL-17, IL-1-beta, MCP-1, MIP-1, IFN-gamma, IL-18, IL-23, CCL-2, CCL-3, CCL-4, and CCL-5.

Conditions Suitable for Treatment with the Defined Peptides

The present invention provides methods of treating or preventing an inappropriate immune response in an individual in need thereof, the method comprising administering a pharmaceutical composition comprising a therapeutically effective amount of a peptide described herein, or a pharmaceutically acceptable salt thereof, to an individual in need of treatment, thereby treating the inappropriate immune response in the individual. Thus, the peptides of the invention find utility in treating a wide range of inflammatory disorders.

The term “individual” or “patient” or “subject” refers herein to a mammal or human which is being treated for an inflammatory condition and/or for an inflammatory condition with visible symptoms. Preferably the individual is a human.

“Therapeutically effective amount” is used herein to denote any amount of a formulation of a composition comprising the peptides which will exhibit an anti-inflammatory effect when applied to the affected area or region of tissue. A single application of the formulations of the present invention may be sufficient, or the formulations may be applied repeatedly over a period of time, such as several times a day for a period of days or weeks. The amount of the active ingredient will vary with the conditions being treated, the stage of advancement of the condition, the age and type of host, and the type and concentration of the formulation being applied. Appropriate amounts in any given instance will be readily apparent to those skilled in the art or capable of determination by routine experimentation.

The terms “treatment” or “treating” of a subject includes the application or administration of a compound of the invention to a subject (or application or administration of a compound of the invention to a cell or tissue from a subject) with the purpose of delaying, slowing, stabilizing, curing, healing, alleviating, relieving, altering, remedying, less worsening, ameliorating, improving, or affecting the disease or condition, the symptom of the disease or condition, or the risk of (or susceptibility to) the disease or condition. The term “treating” refers to any indication of success in the treatment or amelioration of an injury, pathology or condition, including any objective or subjective parameter such as abatement; remission; lessening of the rate of worsening; lessening severity of the disease; stabilization, diminishing of symptoms or making the injury, pathology or condition more tolerable to the subject; slowing in the rate of degeneration or decline; making the final point of degeneration less debilitating; or improving a subject's physical or mental well-being.

As used herein, the terms “preventing”, “prevent” or “prevention” include administering a peptide or composition of the invention to thereby stop or hinder the development of at least one symptom of an inflammatory condition. This term also encompasses treatment of a subject in remission to prevent or hinder relapse.

Inflammatory disorders and related conditions which may be treated or prevented by use of the peptides described herein include, but are not limited to the following: rheumatoid arthritis, bronchitis, contact dermatitis, atopic dermatitis, psoriasis, seborrheic dermatitis, eczema, allergic dermatitis, dermatitis herpetiformis, polymorphous light eruptions, inflammatory dermatoses, folliculitis, alopecia, vitiligo, poison ivy, insect bites, acne inflammation, transplant rejection/tolerance (graft versus host disease, GVHD), irritation induced by extrinsic factors including, but not limited to, chemicals, trauma, pollutants (such as cigarette smoke) and sun exposure, secondary conditions resulting from inflammation including but not limited to xerosis, hyperkeratosis, pruritus, post-inflammatory hyperpigmentation, scarring and the like.

Inflammatory disorders and related conditions which may be treated or prevented by use of the peptides described herein also include those that result in excessive lung inflammation (including when caused by viral or microbial infection), lupus, rheumatoid arthritis, multiple sclerosis, sarcoidosis and scleroderma, hypersensitivity pneumonitis.

Inflammatory disorders and related conditions which may be treated or prevented by use of the peptides described herein also include those that are present during wound healing and inflammatory bowel disease including Ulcerative Colitis and Crohn's Disease.

Preferably, the inflammatory disorders and related conditions which may be treated or prevented using the methods of the invention include contact dermatitis, atopic dermatitis, psoriasis, viral and microbial infections, wounds and inflammatory bowel disease including Crohn's Disease and Ulcerative Colitis, musculosketal inflammatory conditions (including for example, rheumatoid arthritis) and transplant rejection/tolerance (graft versus host disease, GVHD).

### Psoriasis, Dermatitis and Other Inflammatory Skin Disorders

The skilled person will be familiar with methods for identifying individuals requiring prevention or treatment for an inflammatory skin disorder, including psoriasis and dermatitis, and with methods for determining the efficacy of a treatment for such conditions.

As used herein, dermatitis may refer to contact dermatitis atopic dermatitis or dermatitis herpetiformis.

Atopic dermatitis may a form of eczema selected from the group consisting of endogenous eczema, flexural eczema, infantile eczema, and it may also be known as “prurigo Besnier,” “neurodermitis,” or “prurigo diathesique”.

Where the condition is atopic dermatitis, and wherein the peptide of the administered is administered topically, the region of skin contacted with a peptide of the invention may include ruptured and unruptured skin.

Psoriasis is an immune-mediated inflammatory skin disease characterized by skin thickening, red plaques and dry scales. Psoriasis can be triggered by many factors, including injury, trauma, infection and medications. The disease is assessed clinically using the Psoriasis Activity and Severity Index (PASI) scale which ranks severity of erythema (redness), induration (thickness) and desquamation (scale). Study parameters include in-life clinical evaluation of skin, histopathological evaluation of skin sections and optional cytokine analysis in skin and/or internal immune organs. The disease has certain distinct but overlapping clinical phenotypes including chronic plaque lesions, skin eruptions, and pustular lesions. There are several types of psoriasis including Pustular psoriasis, Guttate psoriasis, Inverse psoriasis and Erythrodermic psoriasis. A skilled person will understand that the peptides described herein are suitable for treatment or prevention of all types of psoriasis.

Histologically, the disease is characterized by epidermal thickening due to hyperkeratosis, infiltration of immune cells in dermis and epidermis, parakeratosis and neovascularization. IMQ also causes histopathological changes associated with human disease including; epidermal changes from keratinocyte hyperproliferation and altered differentiation, increased IL-23, immune cell infiltration (T cells, DCs and neutrophils), and altered vascularisation.

The imiquimod-induced psoriasis model is particularly translational into the clinic as it has many of the significant markers of human disease, including histopathology of lesions and strong activation of the immune system. Imiquimod (IMQ) is a ligand for TLRs, for example TLR-7 (Toll-like receptors) of immune cells (including macrophages, monocytes and plasmacytoid dendritic cells), and therefore contributes to strong activation of the immune system. A hallmark of the disease in humans is the involvement of IL-23/IL17 cytokine axis. This imiquimod-induced psoriasis models human plaque-type psoriasis in which the IL-23/IL-17 cytokine axis plays a pivotal role.

Current treatment options for psoriasis include coal tar preparations, corticosteroids, triamcinolone (Acetonide, Trianex), clobetasol (Temovate), synthetic forms of vitamin D, such as calcipotriene and calcitriol (Vectical), retinoids such as tazarotene (Tazorac, Avage), calcineurin inhibitors such as tacrolimus (Protopic) and pimecrolimus (Elidel) medications such as methotrexate, acitretin, cyclosporin and calcipotriol and ultraviolet light therapy.

Thus, it is envisaged that the peptides described herein may be administered to a patient in need in combination with the treatments described above or known in the art for the treatment of psoriasis.

A successful response to treatment may be determined by assessment of the amelioration of one or more symptoms of psoriasis using the Psoriasis Activity and Severity Index (PASI) scale to rank the severity of erythema (redness), induration (thickness) and desquamation (scale), outlined above or by other methods known in the art. For instance, a subject having a positive response may have reduced symptoms of one or more of erythema, induration or desquamation. Such a positive response to treatment may be determined by clinical evaluation of skin, histopathological evaluation of skin sections and optional cytokine analysis in skin and/or internal immune organs. Other means for evaluating a positive response will be within the knowledge of a skilled person.

### Inflammatory Bowel Disease

Inflammatory Bowel Disease (IBD) is a group of chronic inflammatory disorders of the digestive tract. It can develop as ulcerative colitis causing long-lasting inflammation and ulcers in the lining of large intestine and rectum, or as Crohn's disease characterized by inflammation of the lining of digestive tract (particularly the small and large intestine) dispersing into affected tissues such as mouth, esophagus, stomach and the anus.

Crohn's disease is a condition characterised by chronic inflammation in the lining of the digestive system. There may be a small patch of inflammation, or it may spread quite a way along the gut, or there may be several patches in different places. Typical symptoms include recurring diarrhoea, often with a feeling of urgency to get to the toilet, and often with a feeling of wanting to go to the toilet but with nothing to pass, abdominal pain and cramping, which is usually worse after eating, extreme tiredness (fatigue) and/or weight loss. Colonoscopy is used for diagnosis via sampling of small tissue samples (biopsies) for examination under the microscope.

Ulcerative colitis is a chronic inflammatory condition that usually occurs in the rectum (the part of the large bowel that lies just inside the anus) and lower part of the colon, but it may affect the entire large intestine (colon). The colon becomes inflamed and, if this inflammation becomes severe, the lining of the colon is breached and ulcers may form. Diagnosis of ulcerative colitis may be performed via blood test, to check for inflammation, anaemia and protein levels, stool sample, which is checked for infection, X-rays, to help assess the extent of the condition, sigmoidoscopy, to examine the extent of inflammation in the rectum and lower part of the colon and/or colonoscopy, to examine the inside of the entire colon.

### Viral Infections

The peptides and compositions described herein have utility in the treatment or prevention of any viral infection that causes inflammation, including those of the respiratory tract. Such viral infections that are associated with an inflammatory response include the common cold, influenza, coughs and bronchitis, chickenpox, HIV/AIDS, meningitis, pneumonia, herpes, rotavirus, severe acute respiratory syndrome (SARS), severe acute respiratory syndrome coronavirus 2 (SARS-CoV-2) and Middle East respiratory syndrome (MERS).

The skilled person will be familiar with methods for identifying individuals requiring treatment fora viral infection, and with methods for determining the efficacy of a treatment for a given viral infection. For instance, an individual having a viral infection may have one or more symptoms of fever, muscle ache, cough, sore throat and headache. Thus, a positive response to treatment with any peptide or composition described herein includes amelioration of one of more of these symptoms. For instance, an individual having a positive response to treatment with any peptide or composition described herein may have a reduced fever, reduced muscle ache, reduced cough, reduced sore throat or reduced headache, or the symptoms may have disappeared altogether.

Influenza (commonly referred to as “the flu”) is an infectious disease caused by RNA viruses of the family Orthomyxoviridae (the influenza viruses) that affects birds and mammals. The most common symptoms of the disease are chills, fever, sore throat, muscle pains, severe headache, coughing, weakness/fatigue and general discomfort.

Influenza infection involves a multilayered sequence of events. The first is viral infection of the airway and alveolar epithelium and its replication in these cells, during which strategies that limit viral entry or replication can prevent or attenuate the severity of the infection. The second is the innate immune response followed by the adaptive immune response to the virus, which is important for viral clearance but can also induce significant damage to the alveolar epithelium and endothelium. The third is the development of long-term immunity to the infecting viral strain accompanied by the resolution of infiltrates and regeneration of damaged lung tissue, during which there is an increased susceptibility to secondary bacterial infection.

The influenza viruses make up three of the five genera of the family Orthomyxoviridae. Influenza Type A and Type B viruses co-circulate during seasonal epidemics and can cause severe influenza infection. Influenza Type C virus infection is less common but can be severe and cause local epidemics.

Influenza Type A virus can be subdivided into different serotypes or subtypes based on the antibody response to these viruses. Influenza A viruses are divided into subtypes based on two proteins on the surface of the virus: the hemagglutinin (H) and the neuraminidase (N). There are 18 different hemagglutinin subtypes and 11 different neuraminidase subtypes. (H1 through H18 and N1 through N11 respectively.) The sub types that have been confirmed in humans are H1N1, H1N2, H2N2, H3N2, H5N1, H7N2, H7N3, H7N7, H9N2 and H10N7.

In any embodiment, the influenza infection for which treatment, prevention or suppression of the inflammatory response is required is an infection with a virus selected from the group consisting of influenza Types A, B or C.

### Dermatitis

Dermatitis, also known as eczema, is a group of diseases that results in inflammation of the skin including atopic dermatitis, allergic contact dermatitis, irritant contact dermatitis and stasis dermatitis. Although every type of dermatitis has different symptoms, there are certain signs that are common for all of them, including redness of the skin, swelling, itching and skin lesions with sometimes oozing and scarring.

Atopic dermatitis is a chronic inflammatory condition that presents with a red, itchy rash, most commonly where the skin flexes—inside the elbows, behind the knees and the front of the neck. When scratched, the rash can leak fluid and crust over. People with atopic dermatitis may experience improvement and then flare-ups.

Contact dermatitis is an acute or chronic inflammatory skin condition that can be divided into irritant and allergic types. It occurs on areas of the body that have come into contact with substances that either irritate the skin or cause an allergic reaction, such as poison ivy, soap and essential oils. The red rash may burn, sting or itch. Blisters may develop.

Dinitrofluorobenzene (DNFB) is a known irritant that causes contact dermatitis hypersensitivity. Acting as a hapten, it induces a cell-mediated response and causes activation of keratinocytes to produce chemical mediators such as TNF-alpha, IL-1beta and prostaglandin E2, causing migration and maturation of skin dendritic cells. These cytokines then activate vascular endothelial cells to express adhesion molecules (ie ICAM-1 and P/E selectins) which guide T cells to migrate from the blood to tissues.

Haptens such as DNFB also activate mast cells and keratinocytes to produce neutrophil-recruiting chemokines such as CXCL1 and CXCL2, leading to neutrophil recruitment and the innate immune response. The activation of such dendritic cells (ie antigen presenting cells) then leads to the induction of cell-mediated immunity and the infiltration of T cells, leading to the production of IFN-gamma and IL-17. Treg cells regulate contact dermatitis by dampening the leukocyte influx and degrading ATP. B cells and their products (particularly IgM antibodies) are also involved, particularly during the elicitation phase.

Treatments for dermatitis include hydrocortisone cream, gels or ointments, calcineurin inhibitors, antihistamines to relieve severe itching, and monoclonal antibody therapy, including with antibodies which inhibit the activity of IL4/IL-13, IL-33 and TSLP. It is envisaged that the peptides described herein may be administered to a patient in need in combination with the treatments described above or known in the art for the treatment of dermatitis.

### Rheumatoid Arthritis

Rheumatoid arthritis is an autoimmune disease that causes joint pain and swelling as a result of a person's immune system attacking its own tissue. Rheumatoid arthritis is a chronic disease marked by symptoms of inflammation and pain in the joints. These symptoms and signs occur during periods known as flares or exacerbations. Other times are known as periods of remission, where symptoms disappear completely. While rheumatoid arthritis symptoms can affect several organs in the body, the key symptoms of rheumatoid arthritis include joint pain, joint swelling, joint stiffness and loss of joint function and deformities.

Medications used to treat rheumatoid arthritis fall into two main groups. The first group reduces the activity of the immune system that is attacking and damaging healthy joints. These medications are referred to as disease modifying anti-rheumatic drugs (DMARDs) and include methotrexate, sulfasalazine, leflunomide, prednisone and hydroxychloroquine. The second group treat pain and/or inflammation and include medications like paracetamol and anti-inflammatories like ibuprofen or celecoxib.

The peptides of the invention can be used alone or in combination with known medications for treatment of RA.

### Peptides and Suitable Modifications

The present invention establishes utility of a broad class of peptides in the suppression, treatment or prevention of inappropriate inflammatory responses that may or may not be accompanied by an immune response. In a first embodiment, the peptide comprises:
an amino acid sequence of TVLTVV (SEQ ID NO:1), or an amino acid sequence of LLLLLTVLTVV (SEQ ID NO: 2) or functional variants thereof;

wherein the peptide consists of less than 21 amino acid residues, preferably from 6 to 20 amino acid residues, preferably 7, 8, 9, 10, 11, 12, 13, 14, 15, 16, 17, 18, or 19 residues.

As described herein, the inventors believe that a peptide having amino acid sequence of TVLTVV, or an amino acid sequence of LLLLLTVLTVV has an anti-inflammatory effect on symptoms of inflammation and an immune suppressive response as indicated by modifying macrophage and dendritic cell activation, expression of cell surface markers and changes in pro-inflammatory cytokine profiles. In some embodiments, TVLTVV and LLLLLTVLTVV may also each be referred to as an ‘anti-inflammatory domain’ or ‘AID’. According to this embodiment of the invention, the peptide may consist of the sequence of TVLTVV, or LLLLLTVLTVV, or the peptide may comprise TVLTVV, or LLLLLTVLTVV and further comprise additional amino acid residues, provided that the length of the peptide in terms of the number of amino acids is less than 21 amino acids.

As described further herein, additional amino acids (i.e. additional to TVLTVV, or LLLLLTVLTVV) may be residues such as lysine, arginine, or histidine. These may be provided in the form of poly lysine, poly arginine, or poly histidine. These residues may be located N terminal to TVLTVV, or LLLLLTVLTVV (or alternatively located C terminal to TVLTVV, or LLLLLTVLTVV). These residues may function to assist with facilitating the solubilisation of the peptide in a biological fluid or tissue or biologically compatible formulation. Examples of such peptides are shown in Table A.

TABLE A

| Peptide | Sequence |
|---|---|
| SEQ ID NO: 4 | KKKK-TVLTVV |
| SEQ ID NO: 5 | KKKK-LLLLLTVLTVV |
| SEQ ID NO: 24 | RRRR-TVLTVV |
| SEQ ID NO: 25 | RRRR-LLLLLTVLTVV |
| SEQ ID NO: 26 | HHHH-TVLTVV |
| SEQ ID NO: 27 | HHHH-LLLLLTVLTVV |
| SEQ ID NO: 28 | NNNN-TVLTVV |
| SEQ ID NO: 29 | NNNN-LLLLLTVLTVV |

In a second embodiment, the peptide comprises:
a peptide comprising the sequence of SEQ ID NO: 1 or SEQ ID NO: 2;
one or more further amino acids adjacent to the sequence of SEQ ID NO: 1 or SEQ ID NO: 2, provided that the one or more further amino acids do not define the sequence MTPGTQSPFF at a position N terminal adjacent to the sequence of SEQ ID NO: 1 or SEQ ID NO:2 or the sequence TGSGHASSTP at a position C terminal adjacent to the sequence of SEQ ID NO: 1 or SEQ ID NO:2

wherein the peptide comprises at least 12 amino acid residues, preferably from 20 to 100 residues.

According to this embodiment the peptide generally does not consist of the sequence TVLTVV or LLLLLTVLTVV. Rather, the peptide comprises TVLTVV or LLLLLTVLTVV and further comprises additional amino acid residues, other than those defining a sequence of MTPGTQSPFF at a position N terminal adjacent SEQ ID NO: 1 or 2 or the sequence TGSGHASSTP at a position C terminal adjacent to SEQ ID NO: 1 or 2.

Accordingly, the minimum length of the peptide of such a peptide may be 12 amino acid residues although the peptide may be longer and have a length of at least 13, at least 14, at least 15 amino acid residues, or at least 20, 30 40, 50 or up to 1000 residues. The additional amino acids (i.e. additional to TVLTVV, or LLLLLTVLTVV) may be cationic amino acid residues such as lysine, arginine or histidine arranged as described above.

Optionally, the peptide may also comprise a ‘solubilisation domain,’ or a modification which facilitates the solubilisation of the peptide in a biological fluid, tissue or the like. Such improved solubilisation may lead to a higher loading of the peptide in a biological fluid, tissue or cell, thereby improving anti-inflammatory and/or immune suppressive effect of the peptide. The inventors have established such an effect by utilising a poly cationic amino acid sequence. As described further herein, a solubilisation may also comprise poly ethylene glycol (PEG) of varying lengths.

Further still, the peptide may also comprise a molecule in the form of a label described herein (such as biotin or that is conjugated to the peptide to aid in its detection.

Examples of peptides comprising modifications for improving solubility or moieties for enabling detection of the peptide are shown in Table B.

TABLE B

| Peptide | Sequence |
|---|---|
| SEQ ID | KKKK-TPGTQSPFFLLLLLTVLTVV |
| NO: 31 | |
| SEQ ID | (Gd)DOTA-KKKK-PEG-TPGTQSPFFLLLLLTVLTVV |
| NO: 32 | |
| SEQ ID | (Gd)DOTA-KKKK-PEG-TPGTQSPFFLLLLL*TVLTVV, |
| NO: 33 | wherein L* is photoleucine |
| SEQ ID | Biotin-KKKK-PEG-TPGTQSPFFLLLLLTVLTVV |
| NO: 34 | |
| SEQ ID | Biotin-KKKK-PEG-TPGTQSPFFLLLLL*TVLTVV, |
| NO: 35 | wherein L* is photoleucine |
| SEQ ID | KKKK-PGTQSPFFLLLLLTVLTVV |
| NO: 36 | |
| SEQ ID | KKKK-GTQSPFFLLLLLTVLTVV |
| NO: 37 | |
| SEQ ID | KKKK-TQSPFFLLLLLTVLTVV |
| NO: 38 | |
| SEQ ID | KKKK-QSPFFLLLLLTVLTVV |
| NO: 39 | |
| SEQ ID | KKKK-SPFFLLLLLTVLTVV |
| NO: 40 | |
| SEQ ID | KKKK-PFFLLLLLTVLTVV |
| NO: 41 | |
| SEQ ID | KKKK-FFLLLLLTVLTVV |
| NO: 42 | |
| SEQ ID | KKKK-FLLLLLTVLTVV |
| NO: 43 | |
| SEQ ID | KKKK-PEG-MTPGTQSPFFLLLLL*TVLTVV |
| NO: 65 | wherein L* is photoleucine |
| SEQ ID | KKKK-PEG-TPGTQSPFFLLLLL*TVLTVV |
| NO: 66 | wherein L* is photoleucine |

In a further embodiment, the peptide comprises:
an anti-inflammatory peptide comprising or consisting of the sequence of any one of SEQ ID NOs: 1, 2, preferably SEQ ID NO: 3 or 30, or functional variants or fragments thereof);
optionally a modification for facilitating solubilisation of the peptide in a biological fluid, tissue or formulation; and/or a fluorescent label.

According to this embodiment, the peptide does generally does not consist of TVLTVV or LLLLLTVLTVV. Rather, the peptide comprises TVLTVV or LLLLLTVLTVV and further comprises additional amino acid residues to provide the peptide with a length generally greater than 20 amino acids. The further additional amino acids may include the sequence MTPGTQSPFF and/or TGSGHASSTP (or may alternatively include the sequence TPGTQSPFF).

Accordingly, in one example, the peptide comprises or consists of the sequence of SEQ ID NO: 3 (MTPGTQSPFFLLLLLTVLTVV) or comprises or consists of the sequence of SEQ ID NO: 30 (TPGTQSPFFLLLLLTVLTVV), including functional variants and fragments thereof.

The peptide may have a length up to 1000 residues or greater. In this embodiment, additional amino acids (i.e. additional to TVLTVV, or LLLLLTVLTVV) may be cationic amino acid residues such as lysine, arginine or histidine. These amino acids may form or be comprised in a solubilisation domain as described above to facilitate the solubilisation of the peptide in a biological fluid, tissue or formulation. As described further herein, a solubilisation may comprise polyethylene glycol (PEG) of varying lengths. The peptide may also comprise a molecule in the form of a fluorescent label that is conjugated to the peptide to aid in its detection. Examples of such peptides are shown in Table C.

TABLE C

| Peptide | Sequence |
|---|---|
| SEQ ID NO: 44 | KKKK-SEQ ID NO: 64 |
| SEQ ID NO: 45 | PEG-SEQ ID NO: 64 |
| SEQ ID NO: 46 | KKKK-PEG-SEQ ID NO: 64 |
| SEQ ID NO: 47 | AF594-SEQ ID NO: 64 |
| SEQ ID NO: 48 | AF594-PEG-SEQ ID NO: 64 |
| SEQ ID NO: 49 | AF594-KKKK-PEG-SEQ ID NO: 64 |
| SEQ ID NO: 6 | KKKK-MTPGTQSPFFLLLLLTVLTVV |
| SEQ ID NO: 9 | PEG-MTPGTQSPFFLLLLLTVLTVV |
| SEQ ID NO: 12 | KKKK-PEG- |
| | MTPGTQSPFFLLLLLTVLTVV |
| SEQ ID NO: 15 | AF594-MTPGTQSPFFLLLLLTVLTVV |
| SEQ ID NO: 18 | AF594-PEG- |
| | MTPGTQSPFFLLLLLTVLTVV |
| SEQ ID NO: 21 | AF594-KKKK-PEG- |
| | MTPGTQSPFFLLLLLTVLTVV |

In an embodiment, the peptides according to the invention may comprise a linker comprising varying lengths of glycine or serine repeats. The peptide of the invention and the linker may be directly or indirectly linked to either the N or C terminal regions of the peptide. Preferably, the linker is located at the N-terminus of the peptide. In an embodiment, the linker comprises the sequence GGG and a peptide according to the invention comprises or consists of KKKKGGGMTPGTQSPFFLLLLLTVLTVV (SEQ ID NO:22) or KKKKGGGTPGTQSPFFLLLLLTVLTVV (SEQ ID NO:23) functional variants thereof.

The term “peptide” as used herein refers to a compound made up of a single chain of D- or L-amino acids or a mixture of D- and L-amino acids joined by peptide bonds. Generally, peptides contain at least two amino acid residues and are less than about 50 amino acids in length.

The term “protein” as used herein refers to a compound that is composed of linearly arranged amino acids linked by peptide bonds, but in contrast to peptides, has a well-defined tertiary structure. Proteins, as opposed to peptides, generally consist of chains of 50 or more amino acids.

“Polypeptide” as used herein refers to a polymer of at least two amino acid residues and which contains one or more peptide bonds. “Polypeptide” encompasses peptides and proteins, regardless of whether the polypeptide has a well-defined conformation.

It will be understood that in any embodiment, the anti-inflammatory peptide of the invention may be provided in the form of a “functional variant,” “biologically active fragment” or “analog” of the sequence of TVLTVV, or LLLLLTVLTVV. A variant, fragment or analog is a compound that is capable of replicating some or all of the anti-inflammatory and/or immune suppressive effects exemplified for the peptides TVLTVV, or LLLLLTVLTVV as shown in the Examples herein. Functional variants or analogues of the peptides of the invention will generally exhibit at least 70%, 71%, 72%, 73%, 74%, 75%, 76%, 77%, 78%, 79%, 80%, 70%, 81%, 82%, 83%, 84%, 85%, 86%, 87%, 88%, 89%, 90%, 91%, 92%, 93%, 94%, 95%, 96%, 97%, 98%, or at least 99% amino acid identity with all or part of the amino acid sequence of a the peptide. Methods for assaying the capacity of biologically active fragments and analogues to treat inflammatory conditions described herein in the DNFB and imiquimod models are known by a person skilled in the art and include those described herein.

In one embodiment, a functional variant of SEQ ID NO: 1 forming an anti-inflammatory peptide may be as described in Table D.

TABLE D

| Peptide | Sequence |
|---|---|
| SEQ ID NO: 50 | TVLTAA |
| SEQ ID NO: 51 | TLVTLL |
| SEQ ID NO: 52 | TAITII |
| SEQ ID NO: 53 | SVLTVV |
| SEQ ID NO: 54 | SVLSVV |
| SEQ ID NO: 55 | SVLTVV |
| SEQ ID NO: 56 | TVLTVV |

In one embodiment, a functional variant of SEQ ID NO:2 forming an anti-inflammatory peptide may be as described in Table E.

TABLE E

| Peptide | Sequence |
|---|---|
| SEQ ID NO: 57 | WVWTVVTVV |
| SEQ ID NO: 58 | AAAAATVATVV |
| SEQ ID NO: 59 | IIIIITVITVV |
| SEQ ID NO: 60 | LLLLLTLLTLL |
| SEQ ID NO: 61 | LLLLLSVLTVV |
| SEQ ID NO: 62 | LLLLLSVLSVV |
| SEQ ID NO: 63 | LLLLLTVLTVV |

In another embodiment, the peptide according to the invention may comprise a retro-inverso peptide of any peptide described herein. In such a sequence, the D-amino acids represent conformational mirror images of natural L-amino acids.

The peptides of the invention include polymeric forms of amino acids of any length, which can include naturally-occurring amino acids, coded and non-coded amino acids, chemically or biochemically modified, derivatized, or designer amino acids, amino acid analogs, peptidomimetics, and depsipeptides, and polypeptides having modified, cyclic, bicyclic, depsicyclic, or depsibicyclic peptide backbones. They include single chain proteins as well as multimers. They also include conjugated proteins, fusion proteins, including, but not limited to, glutathione S-transferase (GST) fusion proteins, fusion proteins with a heterologous amino acid sequence, fusion proteins with heterologous and homologous leader sequences, fusion proteins with or without N-terminal methionine residues, pegylated proteins, and immunologically tagged, or his-tagged proteins. Also included in the polypeptides of the invention are variations of naturally occurring proteins, where such variations are homologous or substantially similar to the naturally occurring protein, as well as corresponding homologs from different species. Variants of polypeptide sequences include insertions, additions, deletions, or substitutions compared with the subject polypeptides. The polypeptides of the invention also include peptide aptamers.

Protein engineering may be employed to improve or alter the characteristics of the therapeutic peptides of the invention. Recombinant DNA technology known to those skilled in the art can be used to create novel mutant proteins or “muteins” including single or multiple amino acid substitutions, deletions, additions, or fusion proteins. Such modified polypeptides can show desirable properties, such as enhanced activity or increased stability.

For instance, the peptides herein may comprise one or more positively charged amino acids. The positively charged group comprises at least one positively charged amino acid such as arginine (R), lysine (K), or histidine (H). Such examples include KKKK-TVLTVV (SEQ ID NO: 4), KKKK-LLLLLTVLTVV (SEQ ID NO: 5), or KKKK-MTPGTQSPFFLLLLLTVLTVV (SEQ ID NO:6), or functional variants thereof.

In an embodiment, the peptides according to the invention may comprise a solubilising agent. The peptide of the invention and the solubilising agent may be directly or indirectly linked. An example of a solubilising agent is polyethylene (PEG) and in that embodiment the peptide of the invention may be PEGylated. Examples of such peptides include PEG-TVLTVV (SEQ ID NO: 7), PEG-LLLLLTVLTVV (SEQ ID NO: 8), PEG-MTPGTQSPFFLLLLLTVLTVV (SEQ ID NO: 9), KKKK-PEG-TVLTVV (SEQ ID NO: 10), KKKK-PEG-LLLLLTVLTVV (SEQ ID NO: 11), KKKK-PEG-MTPGTQSPFFLLLLLTVLTVV (SEQ ID NO: 12), or functional variants thereof.

In an embodiment, PEG may comprise at least 1, 2, 3, 4, 5, 6, 7, 8, 9, 10, 11, 12, 13, 14, 15 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or more ethylene glycol units. Alternatively, PEG may comprise no ethylene glycol units.

In an embodiment, the peptides according to the invention may comprise a fluorescent tag or label. The peptide of the invention and the fluorescent tag or label may be directly or indirectly linked, and may be at the N-terminus of the peptide. In an embodiment, the fluorescent tag or label is GFP, YFP, BFP, CFP, Y-FAST, AF594, mCherry and dsRed. Examples of such peptides include AF594-TVLTVV (SEQ ID NO: 13) or AF594-LLLLLTVLTVV (SEQ ID NO: 14), AF594-MTPGTQSPFFLLLLLTVLTVV (SEQ ID NO: 15), AF594-PEG-TVLTVV (SEQ ID NO:16), AF594-PEG-LLLLLTVLTVV (SEQ ID NO: 17), AF594-PEG-MTPGTQSPFFLLLLLTVLTVV (SEQ ID NO: 18), AF594-KKKK-PEG-TVLTVV (SEQ ID NO: 19), AF594-KKKK-PEG-LLLLLTVLTVV (SEQ ID NO: 20), AF594-KKKK-PEG-MTPGTQSPFFLLLLLTVLTVV (SEQ ID NO: 21), or functional variants thereof.

In an embodiment, the peptides according to the invention may comprise a linker comprising varying lengths of glycine or serine repeats. The peptide of the invention and the linker may be directly or indirectly linked. Preferably, the linker is located at the N-terminus of the peptide. In an embodiment, the linker comprises the sequence GGG and a peptide according to the invention comprises or consists of KKKKGGGMTPGTQSPFFLLLLLTVLTVV (SEQ ID NO: 22) or functional variants thereof (such as TPGTQSPFFLLLLLTVLTVV having the same modification and linker, i.e., SEQ ID NO: 23: KKKKGGGPGTQSPFFLLLLLTVLTVV).

In addition, the proteins may be purified in higher yields and show better solubility than the corresponding natural polypeptide, at least under certain purification and storage conditions. For many proteins, including the extracellular domain of a membrane associated protein or the mature form(s) of a secreted protein, it is known in the art that one or more amino acids may be deleted from the N-terminus or C-terminus without substantial loss of biological function.

However, even if deletion of one or more amino acids from the N-terminus of a protein results in modification or loss of one or more biological functions of the protein, other biological activities may still be retained. Thus, the ability of the shortened protein to induce and/or bind to antibodies which recognize the complete or mature from of the protein generally will be retained when less than the majority of the residues of the complete or mature protein are removed from the N-terminus. Whether a particular polypeptide lacking N-terminal residues of a complete protein retains its required activity can be determined by routine methods known in the art.

Accordingly, the present invention further provides polypeptides having one or more residues deleted from the amino terminus of the amino acid sequences of the molecules. Similarly, many examples of biologically functional C-terminal deletion mutants are known.

However, even if deletion of one or more amino acids from the C-terminus of a protein results in modification or loss of one or more biological functions of the protein, other biological activities may still be retained. Thus, the ability of the shortened protein to induce and/or bind to antibodies which recognize the complete or mature form of the protein generally will be retained when less than the majority of the residues of the complete or mature protein are removed from the C-terminus. Whether a particular polypeptide lacking C-terminal residues of a complete protein retains such biological activity can be determined by routine methods known in the art.

In addition to terminal deletion forms of the protein discussed above, it also will be recognized by one of ordinary skill in the art that some amino acid sequences of the therapeutic polypeptides of the invention can be varied without significant effect of the structure or function of the protein. If such differences in sequence are contemplated, a skilled person would understand how to determine which domains are critical areas to retain function of the protein.

Such mutants include deletions, insertions, inversions, repeats, and type substitutions, selected according to general rules known in the art, so as to have little effect on activity. For example, guidance on how to make phenotypically silent amino acid substitutions is provided in Bowie et al., (1990) Science 247:1306-1310, wherein the authors indicate that there are two main approaches for studying the tolerance of an amino acid sequence to change. The first method relies on the process of evolution, in which mutations are either accepted or rejected by natural selection.

The second approach uses genetic engineering to introduce amino acid changes at specific positions of a cloned gene and selections, or screens, to identify sequences that maintain functionality. These studies report that proteins are surprisingly tolerant of amino acid substitutions. It is known that certain amino acid changes are likely to be permissive at a certain position of the protein. For example, most buried amino acid residues require nonpolar side chains, whereas few features of surface side chains are generally conserved.

Typically seen as conservative substitutions are the replacements, one for another, among the aliphatic amino acids Ala (A), Val (V), Leu (L), and Ile (I); interchange of the hydroxyl residues Ser (S) and Thr (T), exchange of the acidic residues Asp (D) and Glu (E), substitution between the amide residues Asn (N) and Gln (Q), exchange of the basic residues Lys (K) and Arg (R), and replacements between the aromatic residues Phe (F) and Tyr (Y). Thus, a fragment, derivative, or analog of a polypeptide may be (i) one in which one or more of the amino acid residues are substituted with a conserved or nonconserved amino acid residue; such a substituted amino acid residue may or may not be one encoded by the genetic code; (ii) one in which one or more of the amino acid residues includes a substituent group; (iii) one in which the mature polypeptide is fused with another compound, such as a compound to increase the half-life of the polypeptide (for example, polyethylene glycol); or (iv) one in which the additional amino acids are fused to the above form of the polypeptide, such as an IgG Fc fusion region peptide, a leader or secretory sequence, a sequence employed to purify the above form of the polypeptide, or a proprotein sequence. Such fragments, derivatives, and analogs are deemed to be within the scope of those skilled in the art from the teachings herein.

Thus, the therapeutic polypeptides of the invention may include one or more amino acid substitutions, deletions, or additions, either from natural mutations or human manipulation. As indicated, these changes may be of a minor nature, such as conservative amino acid substitutions, that do not significantly affect the folding or activity of the protein. For instance, the one or more amino acid substitutions, deletions, or additions will not affect the ability of the peptides described herein to suppress, treat or prevent an inappropriate inflammatory response.

Conservative amino acid substitutions include the aromatic substitutions Phe, Trp, and Tyr; the hydrophobic substitutions Leu, Iso, and Val; the polar substitutions Glu and Asp; the basic substitutions Arg, Lys, and His; the acidic substitutions Asp and Glu; and the small amino acid substations Ala, Ser, Thr, Met, and Gly.

Amino acids essential for the functions of the therapeutic polypeptides of the invention can be identified by methods known in the art, such as site-directed mutagenesis or alanine-scanning mutagenesis. The latter procedure introduces single alanine mutations. The resulting mutant molecules are then tested for biological activity such as receptor binding, or in vitro or in vitro proliferative activity.

Further, substitutions of charged amino acids with other charged or neutral amino acids which may produce proteins with highly desirable improved characteristics, such as less aggregation. Aggregation may not only reduce activity but also be problematic when preparing pharmaceutical formulations, because, for example, aggregates can be immunogenic.

Replacing amino acids can also change the selectivity of the binding of a ligand to cell surface receptors. Sites that are critical for ligand-receptor binding can also be determined by structural analysis such as crystallization, nuclear magnetic resonance, or photoaffinity labelling.

The invention also provides nucleic acids or fragments thereof that encode the proteins defined herein comprising a sequence of DNA or RNA, including one having an open reading frame that encodes the therapeutic polypeptide and is capable, under appropriate conditions, of being expressed as one of the therapeutic polypeptides of the instant invention. The term ‘nucleic acid’ also encompasses genomic DNA, cDNA, mRNA, splice variants, antisense RNA, RNAi, DNA comprising one or more single-nucleotide polymorphisms (SNPs), and vectors comprising the subject nucleic acid sequences. Also encompassed in this term are nucleic acids that are homologous or substantially similar or identical to the nucleic acids encoding the therapeutic proteins. Thus, the subject invention provides genes encoding a subject protein, and homologs thereof.

Polynucleotides or nucleic acids of the invention refer to polymeric forms of nucleotides of any length. The polynucleotides can contain deoxyribonucleotides, ribonucleotides, and/or their analogs or derivatives. For example, nucleic acids can be naturally occurring DNA or RNA, or can be synthetic analogs, as known in the art. Polynucleotides of the invention also encompass genomic DNA, genes, gene fragments, exons, introns, regulatory sequences, or regulatory elements, such as promoters, enhancers, initiation and termination regions, other control regions, expression regulatory factors, and expression controls; DNA comprising one or more single-nucleotide polymorphisms (SNPs), allelic variants, isolated DNA of any sequence, and cDNA; mRNA, tRNA, rRNA, ribozymes, splice variants, antisense RNA, antisense conjugates, RNAi, and isolated RNA of any sequence; recombinant polynucleotides, heterologous polynucleotides, branched polynucleotides, labelled polynucleotides, hybrid DNA/RNA, polynucleotide constructs, vectors comprising the subject nucleic acids, nucleic acid probes, primers, and primer pairs.

Polynucleotides of the invention encompass modified nucleic acid molecules, with alterations in the backbone, sugars, or heterocyclic bases, such as methylated nucleic acid molecules, peptide nucleic acids, and nucleic acid molecule analogs, which may be suitable as, for example, probes if they demonstrate superior stability and/or binding affinity under assay conditions. They also encompass single-stranded, double-stranded, and triple helical molecules that are either DNA, RNA, or hybrid DNA/RNA and that may encode a full-length gene or a biologically active fragment thereof.

Polynucleotides of the invention include single nucleotide polymorphisms. Single nucleotide polymorphisms (SNPs) occur frequently in eukaryotic genomes. The nucleotide sequence determined from one individual of a species may differ from other allelic forms present within the population. The present invention encompasses such SNPs. The subject polynucleotides include those that encode variants of the polypeptides described in the instant specification. Thus, in some embodiments, a subject polynucleotide encodes variant polypeptides that include insertions, deletions, or substitutions compared with the polypeptides described herein. Conservative amino acid substitutions include serine/threonine, valine/leucine/isoleucine, asparagine/glutamine, glutamic acid/aspartic acid, etc.

In some embodiments, the proteins or a functional derivative thereof described herein may be modified to enhance suitability for administration, i.e., by the covalent attachment of any type of molecule to the composition such that covalent attachment does not prevent the activity of the composition. For example, but not by way of limitation, derivatives include compositions that have been modified by, inter alia, glycosylation, lipidation, acetylation, pegylation, phosphorylation, amidation, derivatization by known protecting/blocking groups, proteolytic cleavage, linkage to a cellular ligand or other protein, etc. Any of numerous chemical modifications can be carried out by known techniques, including, but not limited to specific chemical cleavage, acetylation, formylation, metabolic synthesis of turicamycin, etc. Additionally, the derivative can contain one or more nonclassical amino acids.

In still other embodiments, the proteins or functional derivatives described herein may be modified to add effector moieties such as chemical linkers, detectable moieties such as for example fluorescent dyes, enzymes, substrates, bioluminescent materials, radioactive materials, and chemiluminescent moieties, or functional moieties such as for example streptavidin, avidin, biotin, a cytotoxin, a cytotoxic agent, and radioactive materials.

In another embodiment, the peptides or functional derivatives described herein may be modified to add metal ion labels such as gadoteric acid which is a macrocycle-structured gadolinium-based MRI contrast agent (GBCA) that includes the organic acid DOTA as a chelating agent, and gadolinium (Gd3+), and is used in form of the meglumine salt (gadoterate meglumine). Such labels are also described herein as (Gd)Dota.

In yet another embodiment, the peptides described herein may contain synthetic variants of amino acid residues that have photoreactivity for observing and characterizing protein-protein interactions (PPD. A non-limiting example is photoleucine, which is a synthetic derivative of the leucine amino acid that is used as its natural analog and is characterized for having photo-reactivity. A skilled person will understand that when a protein containing this amino acid is irradiated with ultraviolet light while interacting with another protein, the complex formed from these two proteins remains attached and can be isolated.

In certain embodiments, any peptide described herein may comprise a cell penetrating peptide (CPP) conjugated to the N- or C-terminal region to facilitate or assist the entry of the peptide into a cell. CPPs are typically amphipathic and cationic peptides of approximately 7 to 30 amino acid residues in length, which facilitate rapid translocation of molecules across a cell membrane. Non-limiting examples of cell penetrating peptides include Tat and peptides based on Tat but it will be appreciated by the skilled person that there are a number of CPPs that could be used in accordance with the invention. Examples of such CPPs are described in the literature, for example in Habault and Poyet, (2019) Molecules, 24: 927; Borerlli et al., (2018) Molecules, 23: 295, incorporated herein by reference.

In certain embodiments, any peptide described herein may be lipidated at the N- or C-terminal region, for example to facilitate or assist the entry of the peptide of the invention into a cell.

Nucleic acids encoding the proteins of the subject invention may be cDNA or genomic DNA or a fragment thereof. The term “gene” shall be intended to mean the open reading frame encoding specific proteins and polypeptides of the subject invention, and introns, as well as adjacent 5′ and 3′ non-coding nucleotide sequences involved in the regulation of expression, up to about 20 kb beyond the coding region, but possibly further in either direction. The gene may be introduced into an appropriate vector for extrachromosomal maintenance or for integration into a host genome. The subject polynucleotides are isolated and obtained in substantial purity, generally as other than an intact chromosome. Usually, the DNA will be obtained substantially free of other nucleic acid sequences that do not include a sequence or fragment thereof of the subject genes, generally being at least about 50%, usually at least about 90% pure and are typically “recombinant,” i.e. flanked by one or more nucleotides with which it is not normally associated on a naturally occurring chromosome.

The invention provides plasmids, i.e., small, independently replicating pieces of extrachromosomal cytoplasmic DNA that can be transferred from one organism to another, comprising the therapeutic polynucleotides of the invention. Plasmids can become incorporated into the genome of a host or can remain independent. Artificially constructed plasmids are commonly used as cloning vectors. The invention also provides vectors, i.e., plasmids that can be used to transfer DNA sequences from one organism to another.

Expression vectors can be used to express the therapeutic gene products of the invention and typically comprise restriction sites to provide for the insertion of nucleic acid sequences encoding heterologous protein or RNA molecules.

Expression vectors may be used to introduce the gene into a cell. Such vectors generally have convenient restriction sites located near the promoter sequence to provide for the insertion of nucleic acid sequences. Transcription cassettes may be prepared comprising a transcription initiation region, the subject gene or fragment thereof, and a transcriptional termination region. The transcription cassettes may be introduced into a variety of vectors, e.g., plasmid; retrovirus, e.g., lentivirus; adenovirus; adeno-associated virus; and the like, where the vectors are able to transiently or stably be maintained in the cells, usually for a period of at least about one day, more usually for a period of at least about several days to several weeks.

### Anti-Inflammatory Agents

In an aspect of the invention, there is provided a composition comprising any peptide described herein and a further anti-inflammatory agent for prevention or treatment of an inflammatory response. A skilled person will be able to readily identify the different types of anti-inflammatory agents that can be used in combination with the peptides described herein. Non-limiting examples of such anti-inflammatory agents include non-selective non-steroidal anti-inflammatory drugs (NSAIDs), selective NSAIDs, corticosteroids, antibodies which exhibit anti-inflammatory properties (eg by inhibiting activity of a pro-inflammatory molecule), and other anti-inflammatory agents known to the skilled person.

Examples of non-selective NSAIDs include diclofenac, ibuprofen, indomethacin, ketoprofen, ketorolac, mefenamic acid, naproxen, piroxicam and sulindac. Examples of selective NSAIDs include celecoxib, etoricoxib, meloxicam and paracoxib.

In a preferred embodiment, there is provided a composition comprising any peptide described herein and a corticosteroid for prevention or treatment of inflammatory responses.

Corticosteroids are synthetic drugs that closely resemble cortisol and work by decreasing inflammation and reducing the activity of the immune system. They are used to treat a variety of inflammatory diseases and conditions including psoriasis, asthma, inflammatory conditions, chronic obstructive pulmonary disease (COPD), hay fever, hives, eczema, vasculitis (inflammation of blood vessels) and myositis (inflammation of muscle), arthritis, inflammatory bowel disease including Crohn's disease, rheumatoid arthritis, lupus, Sjögren's syndrome and gout. Examples of corticosteroid medications include aristocort, decadron, mometasone, cotolone, triamcinolone, cortisone, prednisone, methylprednisolone and betamethasone dipropionate, as used herein.

A skilled person will understand that a corticosteroid used in the invention may be in one of the following forms:

tablets, syrups and liquids—such as prednisolone;

inhalers and nasal sprays—such as beclometasone and fluticasone;

injections (intramuscular, intravenous, intra-articular)—such as methylprednisolone;

ophthalmic—such as eye drops; and

creams, lotions and gels—such as hydrocortisone skin cream.

Corticosteroids described herein or known in the art as are suitable dosages for the administration thereof.

In an embodiment, the use of a corticosteroid in the invention plays a role in treating or preventing an inflammatory response. Thus, in this embodiment, the use of a peptide described herein in combination with a corticosteroid may effectively treat or prevent an inflammatory response. In an embodiment, this effect may be greater than the effect of the peptide alone, and in some embodiments may be synergistic.

The corticosteroid may be administered simultaneously with a peptide of the invention, including when provided in the same or different formulations. Alternatively, the corticosteroid and peptide may be administered at different times, and in different dosage forms (formulations). When provided in different formulations, the peptide and corticosteroid may be administered via the same or different routes of administration.

### Monoclonal Antibodies

The invention also provides compositions comprising any peptide described herein and an antigen binding protein for prevention or treatment of inflammatory responses. Preferably, the antigen binding protein is an antibody or antigen binding fragment thereof. Typically, the antigen binding protein is an antibody, for example, a monoclonal antibody.

In certain embodiments, the antigen binding protein (e.g., monoclonal antibody) for inhibiting the activity of a pro-inflammatory molecule, binds to an anti-inflammatory molecule, thereby inhibiting the activity of the molecule in promoting an inflammatory response. The antigen binding protein may bind to a receptor of the pro-inflammatory molecule, thereby inhibiting the activity of the pro-inflammatory molecule. Non-limiting examples of suitable antigen binding proteins or monoclonal antibodies for use in accordance with the invention include those that are capable of inhibiting the activity of a pro-inflammatory molecule including interleukin-17 (IL-17), interleukin-12 (IL-12), interleukin-6 (IL-6), interleukin-22 (IL-22), interleukin-23 (IL-23), interleukin-36 (IL-36), and tumour necrosis factor-alpha (TNFα). Examples of such antigen binding proteins or monoclonal antibodies include brodalumab, ixekizumab or secukinumab (for inhibiting IL-17), tocilizumab or siltuximab (for inhibiting IL-6), guselkumab or tildrakizumab or mirikizumab or brazikumab or risankizumab or ustekinumab (for inhibiting IL-23), fezakinumab (for inhibiting IL-22), etanercept, infliximab, adalimumab, certolizumab, certolizumab pegol or golimumab (for inhibiting the activity of TNFα).

Methods for generating antibodies are known in the art and/or described in Harlow and Lane (editors) Antibodies: A Laboratory Manual, Cold Spring Harbor Laboratory, (1988). Generally, in such methods an immunogenic fragment or epitope thereof or a cell expressing and displaying same (i.e., an immunogen), optionally formulated with any suitable or desired carrier, adjuvant, or pharmaceutically acceptable excipient, is administered to a non-human animal, for example, a mouse, chicken, rat, rabbit, guinea pig, dog, horse, cow, goat or pig. The immunogen may be administered intranasally, intramuscularly, subcutaneously, intravenously, intradermally, intraperitoneally, or by other known route.

The production of polyclonal antibodies may be monitored by sampling blood of the immunized animal at various points following immunization. One or more further immunizations may be given, if required to achieve a desired antibody titer. The process of boosting and titering is repeated until a suitable titer is achieved. When a desired level of immunogenicity is obtained, the immunized animal is bled and the serum isolated and stored, and/or the animal is used to generate monoclonal antibodies (mAbs).

Monoclonal antibodies are one exemplary form of antibody contemplated by the present invention. The term “monoclonal antibody” or “mAb” refers to a homogeneous antibody population capable of binding to the same antigen(s), for example, to the same epitope within the antigen. This term is not intended to be limited with regard to the source of the antibody or the manner in which it is made.

A suitable antigen binding protein for use in accordance with the present invention may be a synthetic antibody. For example, the antibody may be a chimeric antibody, a humanized antibody, a human antibody synhumanized antibody, primatized antibody or a de-immunized antibody.

The antigen binding protein or antibody, such as a monoclonal antibody, may be in the form of:

(i) a single chain Fv fragment (scFv);

(ii) a dimeric scFv (di-scFv);

(iii) one of (i) or (ii) linked to a constant region of an antibody, Fc or a heavy chain constant domain (CH) 2 and/or CH3, or

(iv) one of (i) or (ii) linked to a protein that binds to an immune effector cell.

Further, as described herein, the antigen binding site or antibody may be in the form of:
(i) a diabody;
(ii) a triabody;
(iii) a tetrabody;
(iv) a Fab;
(v) a F(ab′)2;
(vi) a Fv;
(vii) one of (i) to (vi) linked to a constant region of an antibody, Fc or a heavy chain constant domain (CH) 2 and/or CH3, or
(viii) one of (i) to (vi) linked to a protein that binds to an immune effector cell.

For the production of mAbs any one of a number of known techniques may be used, such as, for example, the procedure exemplified in U.S. Pat. No. 4,196,265 or Harlow and Lane (1988), supra.

For example, a suitable animal is immunized with an immunogen under conditions sufficient to stimulate antibody producing cells. Rodents such as rabbits, mice and rats are exemplary animals. Mice genetically-engineered to express human antibodies, for example, which do not express murine antibodies, can also be used to generate an antibody of the present invention (e.g., as described in WO2002/066630).

Following immunization, somatic cells with the potential for producing antibodies, specifically B lymphocytes (B cells), are selected for use in the mAb generating protocol. These cells may be obtained from biopsies of spleens, tonsils or lymph nodes, or from a peripheral blood sample. The B cells from the immunized animal are then fused with cells of an immortal myeloma cell, generally derived from the same species as the animal that was immunized with the immunogen.

Hybrids are amplified by culture in a selective medium comprising an agent that blocks the de novo synthesis of nucleotides in the tissue culture media. Exemplary agents are aminopterin, methotrexate and azaserine.

The amplified hybridomas are subjected to a functional selection for antibody specificity and/or titer, such as, for example, by flow cytometry and/or immunohistochemistry and/or immunoassay (e.g. radioimmunoassay, enzyme immunoassay, cytotoxicity assay, plaque assay, dot immunoassay, and the like).

Alternatively, ABL-MYC technology (NeoClone, Madison Wis. 53713, USA) is used to produce cell lines secreting MAbs (e.g., as described in Largaespada et al, J. Immunol. Methods. 197: 85-95, 1996).

Antibodies can also be produced or isolated by screening a display library, e.g., a phage display library, e.g., as described in U.S. Pat. No. 6,300,064 and/or U.S. Pat. No. 5,885,793. For example, the present inventors have isolated fully human antibodies from a phage display library.

Suitable dosages of an antigen binding protein or antibody as described herein will vary depending on the specific an antigen binding site, the condition to be treated and/or the subject being treated. It is within the ability of a skilled physician to determine a suitable dosage, e.g., by commencing with a sub-optimal dosage and incrementally modifying the dosage to determine an optimal or useful dosage. Alternatively, to determine an appropriate dosage for treatment/prophylaxis, data from the cell culture assays or animal studies are used, wherein a suitable dose is within a range of circulating concentrations that include the ED₅₀ of the active compound with little or no toxicity. The dosage may vary within this range depending upon the dosage form employed and the route of administration utilized. A therapeutically/prophylactically effective dose can be estimated initially from cell culture assays. A dose may be formulated in animal models to achieve a circulating plasma concentration range that includes the IC₅₀ (i.e., the concentration or amount of the compound which achieves a half-maximal inhibition of symptoms) as determined in cell culture. Such information can be used to more accurately determine useful doses in humans. Levels in plasma maybe measured, for example, by high performance liquid chromatography.

### Pharmaceutical Compositions and Routes of Administration

Provided herein is a pharmaceutical composition comprising a pharmaceutically effective amount of a peptide described herein. In additional embodiments there is provided a pharmaceutical composition comprising a pharmaceutically acceptable salt.

The term “pharmaceutically acceptable salt” also refers to a salt of the compositions of the present invention having an acidic functional group, such as a carboxylic acid functional group, and a base. Pharmaceutically acceptable salts include, by way of non-limiting example, may include sulfate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate, camphorsulfonate, pamoate, phenylacetate, triftuoroacetate, acrylate, chlorobenzoate, dinitrobenzoate, hydroxybenzoate, methoxybenzoate, methylbenzoate, o-acetoxybenzoate, naphthalene-2-benzoate, isobutyrate, phenylbutyrate, a-hydroxybutyrate, butyne-1,4-dicarboxylate, hexyne-1,4-dicarboxylate, caprate, caprylate, cinnamate, glycolate, heptanoate, hippurate, malate, hydroxymaleate, malonate, mandelate, mesylate, nicotinate, phthalate, teraphthalate, propiolate, propionate, phenylpropionate, sebacate, suberate, p-brornobenzenesulfonate, chlorobenzenesulfonate, ethylsulfonate, 2-hydroxyethylsulfonate, methylsulfonate, naphthiene-1-sulfonate, naphthalene-2-sulfonate, naphthiene-1,5-sulfonate, xylenesulfonate, and tartarate salts.

Further, the peptides or functional variants described herein can be administered to a subject as a component of a composition that comprises a pharmaceutically acceptable carrier or vehicle. Such compositions can optionally comprise a suitable amount of a pharmaceutically acceptable excipient so as to provide the form for proper administration.

Pharmaceutical excipients can be liquids, such as water and oils, including those of petroleum, animal, vegetable, or synthetic origin, such as peanut oil, soybean oil, mineral oil, sesame oil and the like. The pharmaceutical excipients can be, for example, saline, gum acacia, gelatin, starch paste, talc, keratin, colloidal silica, urea and the like. In addition, auxiliary, stabilizing, thickening, lubricating, and coloring agents can be used.

In one embodiment, the pharmaceutically acceptable excipients are sterile when administered to a subject. Water is a useful excipient when any agent described herein is administered intravenously. Saline solutions and aqueous dextrose and glycerol solutions can also be employed as liquid excipients, specifically for injectable solutions. Suitable pharmaceutical excipients also include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. Any agent described herein, if desired, can also comprise minor amounts of wetting or emulsifying agents, or pH buffering agents.

In one embodiment, the peptides or functional variants described herein can take the form of solutions, suspensions, emulsion, drops, tablets, pills, pellets, capsules, capsules containing liquids, powders, sustained-release formulations, suppositories, emulsions, aerosols, sprays, suspensions, nanoparticles or microneedles or any other form suitable for use. In one embodiment, the composition is in the form of a capsule. Other examples of suitable pharmaceutical excipients are described in Remington's Pharmaceutical Sciences 1447-1676 (Alfonso R. Gennaro eds., 19th ed. 1995), incorporated herein by reference.

Where necessary, the peptides or functional variants described herein also includes a solubilizing agent. Also, the agents can be delivered with a suitable vehicle or delivery device as known in the art.

The peptides outlined herein can be co-delivered in a single delivery vehicle or delivery device. Compositions for administration can optionally include a local anesthetic such as, for example, lignocaine to lessen pain at the site of the injection.

The formulations comprising the peptides or functional variants described herein, or a corticosteroid or monoclonal antibody described herein, may conveniently be presented in unit dosage forms and may be prepared by any of the methods well known in the art. Such methods generally include the step of bringing the therapeutic agents into association with a carrier, which constitutes one or more accessory ingredients. Typically, the formulations are prepared by uniformly and intimately bringing the therapeutic agent into association with a liquid carrier, a finely divided solid carrier, or both, and then, if necessary, shaping the product into dosage forms of the desired formulation (e.g., wet or dry granulation, powder blends, etc., followed by tableting using conventional methods known in the art).

In one embodiment, the peptides or functional variants described herein, or a corticosteroid or monoclonal antibody described herein, is formulated in accordance with routine procedures as a composition adapted for a mode of administration described herein. In one aspect, the pharmaceutical composition is formulated for administration to the respiratory tract, the skin or the gastrointestinal tract. Accordingly, the pharmaceutical composition comprising the peptides described herein for administration to the respiratory tract may be formulated as an inhalable substance, such as common to the art and described herein. In another embodiment, the pharmaceutical composition comprising the peptides described herein for administration to the gastrointestinal tract may be formulated with an enteric coating, such as common to the art and described herein. In yet another embodiment, the pharmaceutical composition comprising the peptides described herein for administration to the skin comprises a base that allows for absorption of the composition into the dermal layers of an individual in need thereof.

In an embodiment, the pharmaceutical composition may be administered in a single or as multiple doses. The pharmaceutical composition may be administered between one to three times in a 24 hour period, or daily over a 7 day period or longer. The frequency and timing of administration may be as described in the Examples.

Routes of administration include, for example: intradermal, intramuscular, intraperitoneal, intravenous, subcutaneous, intranasal, epidural, oral, sublingual, intracerebral, intra-lymph node, intratracheal, intra-articular, intravaginal, transdermal, rectally, by inhalation, or topically, particularly to the ears, nose, eyes, or skin. In some embodiments, the administering is effected orally or by parenteral injection. The mode of administration can be left to the discretion of the practitioner, and depends in-part upon the site of the medical condition. In most instances, administration results in the release of any agent described herein into the bloodstream.

The peptides disclosed herein can be dosed at a total amount of about 0.2 to about 2400 mg per day. The dosage can be divided into two or three doses over the day or given in a single daily dose. Specific amounts of the total daily amount of the therapeutic contemplated for the disclosed methods include about 0.2 mg, about 0.5 mg, about 5 mg, about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, about 150 mg, about 200 mg, about 250 mg, about 267 mg, about 300 mg, about 350 mg, about 400 mg, about 450 mg, about 500 mg, about 534 mg, about 550 mg, about 600 mg, about 650 mg, about 700 mg, about 750 mg, about 800 mg, about 850 mg, about 900 mg, about 950 mg, about 1000 mg or higher.

In some embodiments, the patient or individual is a mammal, e.g., a human, mouse, rat, guinea pig, dog, cat, horse, cow, pig, rabbit, sheep, or non-human primate, such as a monkey, chimpanzee, or baboon. In other embodiments, the subject and/or animal is a non-mammal, such, for example, a zebrafish. In some embodiments, the subject and/or animal may comprise fluorescently-tagged cells (with e.g. GFP). In some embodiments, the subject and/or animal is a transgenic animal comprising a fluorescent cell.

In some embodiments, the patient or individual is a human. In some embodiments, the human is a paediatric human. In other embodiments, the human is an adult human. In other embodiments, the human is a geriatric human. In other embodiments, the human may be referred to as a patient.

In certain embodiments, the human has an age in a range of from about 0 months to about 6 months old, from about 6 to about 12 months old, from about 6 to about 18 months old, from about 18 to about 36 months old, from about 1 to about 5 years old, from about 5 to about 10 years old, from about 10 to about 15 years old, from about 15 to about 20 years old, from about 20 to about 25 years old, from about 25 to about 30 years old, from about 30 to about 35 years old, from about 35 to about 40 years old, from about 40 to about 45 years old, from about 45 to about 50 years old, from about 50 to about 55 years old, from about 55 to about 60 years old, from about 60 to about 65 years old, from about 65 to about 70 years old, from about 70 to about 75 years old, from about 75 to about 80 years old, from about 80 to about 85 years old, from about 85 to about 90 years old, from about 90 to about 95 years old or from about 95 to about 100 years old.

In other embodiments, the subject is a non-human animal, and therefore the invention pertains to veterinary use. In a specific embodiment, the non-human animal is a household pet. In another specific embodiment, the nonhuman animal is a livestock animal.

### Enteric Application

In an embodiment, there is provided a method of treating or preventing an inappropriate inflammatory response in the gastrointestinal tract of an individual, the method comprising administering a pharmaceutical composition comprising a pharmaceutically effective amount of a peptide described herein, or a pharmaceutically acceptable salt thereof, to an individual in need of treatment, thereby treating or preventing the inappropriate inflammatory response in the gastrointestinal tract of the individual.

A positive response to therapy may be prevention or attenuation of worsening of inflammatory bowel symptoms e.g. reduction in the levels of inflammatory erythema or cytokine/chemokine expression, reduction in ulceration, intestinal discomfort and pain.

The compositions provided herein may be formulated by a variety of methods apparent to those of skill in the art of pharmaceutical formulation. The various release properties described above may be achieved in a variety of different ways. Suitable formulations include, for example, tablets, capsules, press coat formulations, and other easily administered formulations.

Suitable pharmaceutical formulations may contain, for example, from about 0.1% to about 99.9%, preferably from about 1% to about 60%, of the active ingredient(s). Pharmaceutical formulations for the combination therapy for enteral or parenteral administration are, for example, those in unit dosage forms, such as sugar-coated tablets, tablets, capsules or suppositories, or ampoules. If not indicated otherwise, these are prepared in a manner known per se, for example by means of conventional mixing, granulating, sugar-coating, dissolving or lyophilizing processes. It will be appreciated that the unit content of a combination partner contained in an individual dose of each dosage form need not in itself constitute an effective amount since the necessary effective amount may be reached by administration of a plurality of dosage units.

### Respiratory Application

In an embodiment, there is provided a method of treating or preventing an inappropriate inflammatory response in the respiratory tract of an individual, the method comprising administering a pharmaceutical composition comprising a pharmaceutically effective amount of a peptide described herein, or a pharmaceutically acceptable salt thereof, to an individual in need of treatment, thereby treating or preventing the inappropriate inflammatory response in the respiratory tract of the individual.

The term ‘respiratory’ refers to the process by which oxygen is taken into the body and carbon dioxide is discharged, through the bodily system including the nose, throat, larynx, trachea, bronchi and lungs.

The term ‘respiratory disease’ or ‘respiratory condition’ refers to any one of several ailments that involve inflammation and affect a component of the respiratory system including the upper (including the nasal cavity, pharynx and larynx) and lower respiratory tract (including trachea, bronchi and lungs).

A symptom of respiratory disease may include cough, excess sputum production, a sense of breathlessness or chest tightness with audible wheeze. Exercise capacity may be quite limited. The impact of each of these conditions may also be measured by days of lost work/school, disturbed sleep, requirement for bronchodilator drugs, requirement for glucocorticoids including oral glucocorticoids.

The existence of, improvement in, treatment of or prevention of a respiratory disease may be determined by any clinically or biochemically relevant method of the subject or a biopsy therefrom. For example, a parameter measured may be the presence or degree of lung function, signs and symptoms of obstruction; exercise tolerance; night time awakenings; days lost to school or work; bronchodilator usage; inhaled corticosteroid (ICS) dose; oral (glucocorticoid) GC usage; need for other medications; need for medical treatment; hospital admission.

A positive response to therapy may also be prevention or attenuation of worsening of respiratory symptoms eg reduction in the levels of inflammatory erythema or cytokine/chemokine expression, reduction in coughing or sputum production, or a reduction in chest tightness.

The peptides as described herein may be in compositions formulated for administration to the upper respiratory tract (URT), the lower respiratory tract (LRT) or to both the URT and LRT. The peptides as described herein may be formulated for intranasal administration, including dry powder, sprays, mists, or aerosols. This may be particularly preferred for treatment of a respiratory infection. Suitable formulations, wherein the carrier is a liquid, for administration, as for example, a nasal spray or as nasal drops, include aqueous or oily solutions of the active ingredient. Alternatively, the composition may be a dry powder, including for example excipients e.g. mannitol, trehalose, lactose and administered to the upper respiratory tract. A liquid or dry powder formulation may be designed for upper respiratory tract and lower respiratory tract delivery.

Other ingredients, such as art known preservatives, colorants, lubricating or viscous mineral or vegetable oils, perfumes, natural or synthetic plant extracts such as aromatic oils, and humectants and viscosity enhancers such as, e.g., glycerol, can also be included to provide additional viscosity, moisture retention and a pleasant texture and odour for the formulation. For nasal administration of solutions or suspensions according to the invention, various devices are available in the art for the generation of drops, droplets and sprays. For example, a compound or composition described herein can be administered into the nasal passages by means of a simple dropper (or pipet) that includes a glass, plastic or metal dispensing tube from which the contents are expelled drop by drop by means of air pressure provided by a manually powered pump, e.g., a flexible rubber bulb, attached to one end.

The tear secretions of the eye drain from the orbit into the nasal passages, thus, if desirable, a suitable pharmaceutically acceptable ophthalmic solution can be readily provided by the ordinary artisan as a carrier for the peptides or composition described herein to be delivered and can be administered to the orbit of the eye in the form of eye drops to provide for both ophthalmic and intranasal administration.

In one embodiment, a premeasured unit dosage dispenser that includes a dropper or spray device containing a solution or suspension for delivery as drops or as a spray is prepared containing one or more doses of the drug to be administered. The invention also includes a kit containing one or more unit dehydrated doses of compound, together with any required salts and/or buffer agents, preservatives, colorants and the like, ready for preparation of a solution or suspension by the addition of a suitable amount of water. The water may be sterile or nonsterile, although sterile water is generally preferred.

### Application to Skin

In an embodiment, there is provided a method of treating or preventing an inappropriate inflammatory response in the dermal layers or skin of an individual (including epidermis, hypodermis/subcutaneous tissue), the method comprising administering a peptide, pharmaceutical composition comprising a pharmaceutically effective amount of a peptide described herein, or a pharmaceutically acceptable salt thereof, to an individual in need of treatment, thereby treating or preventing the inappropriate inflammatory response in the dermal layers or skin of an individual.

A positive response to therapy may be prevention or attenuation of worsening of skin symptoms eg reduction in the levels of inflammatory erythema or cytokine/chemokine expression, reduction in swelling, rash, scaling and/or fluid seepage from the affected area.

A pharmaceutical composition suitable for the treatment of an inappropriate inflammatory response in the skin may be formulated with one or more excipients. Additionally, the pharmaceutical composition may also be administered along with a steroid cream, moisturizer, coal tar, cream or ointment or retinoid creams.

Other substances, such as biologically active agents, pharmaceutical excipients, and cosmetic agents may be included in the topical compositions of this invention.

Biologically active agents may include, but are not limited to, flavanoid/flavone compounds which include but are not limited to tanetin, 3,7,3′-trimethoxyquercetagetin, apigenin and its derivatives. When flavanoid/flavone compounds are present, they are present at a concentration of between about 0.001% to about 0.5% preferably, between about 0.005% and 0.2% based on the weight of the topical composition.

Additional biologically active agents include but are not limited to sunscreens, anti-wrinkling/antiaging agents, antifungal agents, antibiotic agents, anti-acne and antipsoriatic agents, depigmentating agents, where such agents may be utilized so long as they are physically and chemically compatible with the other components of the topical composition.

The compositions of this invention may include additional skin actives. Actives can be but not limited to vitamin compounds. Skin lightening agents (kojic acid, ascorbic acid and derivatives such as ascorbyl pamiltate, and the like); anti-oxidant agents such as tocopherol and esters; nicotinamide, metal chelators, retinoids and derivatives, moisturizing agents, hydroxy acids such as salicylic acid, sun screen such as octyl methoxycinnamate, oxybenzone, avobenzone, and the like, sun blocks such as titanium oxide and zinc oxide, and skin protectants. Mixtures of above skin actives may be used.

Sunscreens which may be used in the compositions of this invention may include but are not limited to organic or inorganic sunscreens, such as, octylmethoxycinnamate and other cinnamate compounds, titanium dioxide, zinc oxide and the like.

Antifungal agents include but are not limited to miconazole, econazole, ketoconazole, itraconazole, fluconazole, bifoconazole, terconazole, butoconazole, tioconazole, oxiconazole, sulconazole, saperconazole, clotrimazole, undecylenic acid, haloprogin, butenafine, tolnaftate, nystatin, ciclopirox olamine, terbinafine, amorolfine, naftifine, elubiol, griseofulvin, and their pharmaceutically acceptable salts.

Antibiotic (or antiseptic agents) include but are not limited to mupirocin, neomycin sulfate, bacitracin, polymyxin B, I-ofloxacin, tetracyclines (chlortetracycline hydrochloride, oxytetracycline hydrochloride and tetrachcycline hydrochoride), clindamycin phosphate, gentamicin sulfate, benzalkonium chloride, benzethonium chloride, hexylresorcinol, methylbenzethonium chloride, phenol, quaternary ammonium compounds, triclocarbon, triclosan, tea tree oil, benzoyl peroxide and their pharmaceutically acceptable salts.

Acne ingredients include but are not limited to agents that normalize epidermal differentiation (e.g. retinoids), keratolytic agents (e.g. salicylic acid and alpha hydroxy acids), benzoyl peroxide, antibiotics and compounds or plant extracts that regulate sebum.

Antipsoriatic agents include but are not limited to corticosteroids (e.g., betamethasone dipropionate, betamethasone valerate, clobetasol propionate, diflorasone diacetate, halobetasol propionate, amcinonide, desoximetasone, fluocinonide, fluocinolone acetonide, halcinonide, triamcinolone acetate, hydrocortisone, hydrocortisone valerate, hydrocortisone butyrate, aclometasone dipropionte, flurandrenolide, mometasone furoate, methylprednisolone acetate), Vitamin D and its analogues (e.g. calcipotriene), retinoids (e.g. Tazarotene) and anthraline.

Cosmetic agents which may be used in the compositions of this invention may include, but are not limited to those agents which prevent potential skin irritation, such as emollients, vitamins and antioxidants (e.g., vitamin E) and herbal extracts (e.g., aloe vera). Further, the cosmetic agents may include humectants, antioxidants/preservatives, plant extracts, flavors, fragrances, surface active agents, and the like. Examples of humectants include glycerol, sorbitol, propylene glycol, ethylene glycol, 1,3-butylene glycol, polypropylene glycol, xylitol, maltitol, lactitol, oat protein, allantoin, acetamine MEA, hyaluronic acid and the like. They may be used either singly or in combination.

Cosmetic agents may also include substances which mask the symptoms of inflammatory disorders and related conditions; such substances include but are not limited to pigments, dyes, and other additives (e.g., silica, talk, zinc oxide, titanium oxide, clay powders). The pharmaceutical excipients include but are not limited to pH modifying agents such as pH-modifying agents, organic solvents (e.g., propylene glycol, glycerol, etc.), cetyl alcohol, kaolin, talc, zinc oxide, titanium oxide, cornstarch, sodium gluconate, oils (e.g., mineral oil), ceteareth-20, ceteth-2, surfactants and emulsifiers, thickener (or binders), perfume, antioxidants, preservatives, and water.

Binders or thickeners may be used in the compositions of this invention to provide substantivity and physical stability to the compositions. Binders or thickeners suitable for use in the compositions of this invention include cellulose derivatives such as alkali metal salts of carboxymethylcellulose, methyl cellulose, hydroxyethyl cellulose and sodium carboxymethylhydroxyethyl cellulose, alkali metal alginates such as sodium alginate, propylene glycol alginate, gums such as carrageenan, xanthan gum, tragacanth gum, caraya gum and gum arabic, and synthetic binders such as polyvinyl alcohol, polysodium acrylate and polyvinyl pyrrolidone. Thickeners such as natural gums and synthetic polymers, as well as coloring agents and fragrances also are commonly included in such compositions.

Examples of preservatives which may be used in the compositions of this invention include, but are not limited to, salicylic acid, chlorhexidine hydrochloride, phenoxyethanol, sodium benzoate, methyl para-hydroxybenzoate, ethyl para-hydroxybenzoate, propyl para-hydroxybenzoate, butyl parahydroxybenzoate and the like.

Examples of flavors and fragrances which may be used in the compositions of this invention include menthol, anethole, carvone, eugenol, limonene, ocimene, n-decylalcohol, citronellol, a-terpineol, methyl salicylate, methyl acetate, citronellyl acetate, cineole, linalool, ethyl linalool, vanillin, thymol, spearmint oil, peppermint oil, lemon oil, orange oil, sage oil, rosemary oil, cinnamon oil, pimento oil, cinnamon leaf oil, perilla oil, wintergreen oil, clove oil, eucalyptus oil and the like.

The compositions of the present invention may be prepared in a number of forms for topical application to a patient. For example, the composition may be applied in a gel, cream, ointment, shampoo, scalp conditioners, liquid, spray liquid, paint-/brush-on preparation, aerosol, powder or adhesive bandage. In addition the composition may be impregnated on a bandages, hydrocolloid or other wound dressing, treatment patch or on cloth wipe products, such as baby wipes or facial wipes.

The compositions of this invention may be in the form of emulsions, such as creams, lotions and the like. Such compositions may have more than one phase and may include surface active agents which enable multiphase emulsions to be manufactured.

Examples of surface active agents which may be used in the compositions of this invention include sodium alkyl sufates, e.g., sodium lauryl sulfate and sodium myristyl sulfate, sodium N-acyl sarcosinates, e.g., sodium N-lauroyl sarcosinate and sodium N-myristoyl sarcosinate, sodium dodecylbenzenesulfonate, sodium hydrogenated coconut fatty acid monoglyceride sulfate, sodium lauryl sulfoacetate and N-acyl glutamates, e.g., N-palmitoyl glutamate, N-methylacyltaurin sodium salt, N-methylacylalanine sodium salt, sodium a-olefin sulfonate and sodium dioctylsulfosuccinate; N-alkylaminoglycerols, e.g., N-lauryldiaminoethylglyecerol and N-myristyldiaminoethylglycerol, N-alkyl-N-carboxymethylammonium betaine and sodium 2-alkyl-1-hydroxyethylimidazoline betaine; polyoxyethylenealkyl ether, polyoxyethylenealkylaryl ether, polyoxyethylenelanolin alcohol, polyoxyethyleneglyceryl monoaliphatic acid ester, polyoxyethylenesorbitol aliphatic acid ester, polyoxyethylene aliphatic acid ester, higher aliphatic acid glycerol ester, sorbitan aliphatic acid ester, Pluronic type surface active agent, and polyoxyethylenesorbitan aliphatic acid esters such as polyoxyethylenesorbitan monooleate and polyoxyethylenesorbitan monolaurate. Emulsifier-type surfactants know to those of skill in the art should be used in the compositions of this invention.

Another aspect of the present invention is a dermatologically acceptable carrier. Such a suitable carrier is adequate for topical use. It is not only compatible with the active ingredients described herein, but will not introduce any toxicity and safety issues. An effective and safe carrier varies from about 50% to about 99% by weight of the compositions of this invention, more preferably from about 75% to about 99% of the compositions and most preferably from about 85% to about 95% by weight of the compositions.

The choice of which pharmaceutical excipient or biological agent, or cosmetic agent to use is often controlled or affected by the type of inflammatory disorder or related condition which is being treated. For example, if the compositions of this invention were used to treat a skin inflammation associated with athlete's foot, jock itch or diaper rash, talc would be a preferred pharmaceutical excipient and an antifungal agents would be preferred biological agents. If the compositions of this invention were to be used to treat eczema of the scalp, emulsifiers and oils would be preferred pharmaceutical excipients.

### Kits

The invention provides kits that can simplify the administration of any agent described herein. An exemplary kit of the invention comprises any composition described herein in unit dosage form. In one embodiment, the unit dosage form is a container, such as a pre-filled syringe, which can be sterile, containing any agent described herein and a pharmaceutically acceptable carrier, diluent, excipient, or vehicle. The kit can further comprise a label or printed instructions instructing the use of any agent described herein. The kit may also include a lid speculum, topical anaesthetic, and a cleaning agent for the administration location. The kit can also further comprise one or more additional agent described herein. In one embodiment, the kit comprises a container containing an effective amount of a composition of the invention and an effective amount of another composition, such those described herein.

The present invention additionally comprises a kit comprising one or more of the following:

(i) a peptide described herein;

(ii) an antigen binding site of the invention;

(iii) an anti-inflammatory agent described herein;

(iii) a pharmaceutical composition of the invention.

In an embodiment, the kit can additionally comprise a detection means, e.g., linked to a peptide of the invention.

In the case of a kit for therapeutic/prophylactic use, the kit can additionally comprise a pharmaceutically acceptable carrier.

Optionally a kit of the invention is packaged with instructions for use in a method described herein according to any example.

It will be understood that the invention disclosed and defined in this specification extends to all alternative combinations of two or more of the individual features mentioned or evident from the text or drawings. All of these different combinations constitute various alternative aspects of the invention.

### Example 1: Production of Peptides Suitable for Use in the Invention

### Synthesis of RP23 (SEQ ID NO:12).

H-KKKK-CH₂CH₂OCH₂CH₂OCH₂CO-MTPGTQSPFFLLLLLTVLTVV-OH RP23 (Peptide 12) was synthesised by automated SPPS on Chemmatrix® CTC resin on a 12.5 μmol scale with coupling reactions performed at 50° C. At both Leu-Thr junctions, a pseudoproline dipeptide Fmoc-L-Leu-L-Thr[ψMe,MePro]-0H was incorporated. The peptide was cleaved from resin with TFA:iPr₃SiH:thioanisole:H₂O (90:2.5:2.5:5 v/v/v/v). Preparative HPLC purification followed by lyophilisation afforded RP23 (peptide 12) as a white solid (9.6 mg, 26% yield). LRMS calcd: [M+2H]²⁺=1475.38, [M+3H]³⁺=983.92, [M+4H]⁴⁺=738.19; found (ESI+) 1145.90, 764.10. HRMS calcd for C₁₄₀H₂₃₉N₃₁O₃₅S: [M+H]⁺=2946.760; found (MALDI-TOF): 2946.758.

### Example 2: Effect of Peptide Administration on Local Suppression of the Immune System

### Contact Hypersensitivity Model—Methodology

The abdomens of 8-week old female C57BL/6 mice (Australian BioResources) were coarse shaved. A minimum of 24 hours later, RP23 (SEQ ID NO:12) was delivered via sub-cutaneous injection at the abdomen in 50 μl phosphate buffered saline. Seven days later, mice were topically sensitised to 1-fluoro-2,4-dinitrobenzene (DNFB, 0.5 vol %) in olive oil/acetone (1:4, v/v, 30 μl) via application to the shaved abdomen. Five days after sensitisation, mice were challenged on the ears with topical DNFB (0.25 vol %) in olive oil/acetone (1:4, v/v, 15 μl per ear). Ear thickness was measured daily using manual callipers from immediately before challenge, up to 96 hours post challenge. Positive controls for ear swelling consisted of mice that were injected with PBS instead of RP23. Negative or irritant controls consisted of a group of mice that received the DNFB challenge, but not the sensitisation. The average of the change in ear thickness from this control group was subtracted from the change in ear thickness of other samples to account for non-specific irritation due to the application of DNFB, olive oil and acetone.

### Imiquimod-Induced Psoriasis Model—Methodology

The backs of 8-week old female C57BL/6 mice (Australian BioResources) were coarse shaved. A minimum of 24 hours later, 10 nmol RP23 was delivered via sub-cutaneous injection at the lower back in 200 μl phosphate buffered saline (PBS). Groups of mice acting as positive controls for disease development received injection of PBS only. One or seven days later, psoriasis-like inflammation was induced via topical application of 5% imiquimod cream (Aldara, Inova Pharmaceuticals) to the shaved back (57.5 mgs) and one ear (5 mg, divided between dorsal and ventral sides). The contralateral ear served as a negative control for imiquimod-induced inflammation. Imiquimod application was repeated daily for four consecutive days. C57BL/6 mice experience rapid weight loss (up to 20%) upon application of imiquimod cream. Supportive care was provided in the form of dietary supplementation commenced 2 days prior to imiquimod application, and intra-peritoneal injection of 300 μl sterile PBS on days two and three of imiquimod application. Disease progression was monitored for five days, by recording weight loss and grading disease of the back skin using a modified Psoriasis Area and Severity Index (PASI) clinical scoring system, that assessed only disease severity. Additionally, erythema meter (DSM II ColorMeter, Cortex Technology, Denmark) readings were taken at four quadrants on the back skin and skin backfold thickness measurements were recorded using manual callipers. Disease progression of the ear skin was determined by recording ear thickness, measured by manual callipers. As a positive control for immune suppressive therapy, groups of mice received daily topical application of betamethasone dipropionate 0.05% (MSD) cream (30 mgs on the back, 10 mgs on the ear). Mice receiving steroid therapy experienced a greater degree of weight loss, and substantial reductions in spleen weight, indicative of systemic immune suppression.

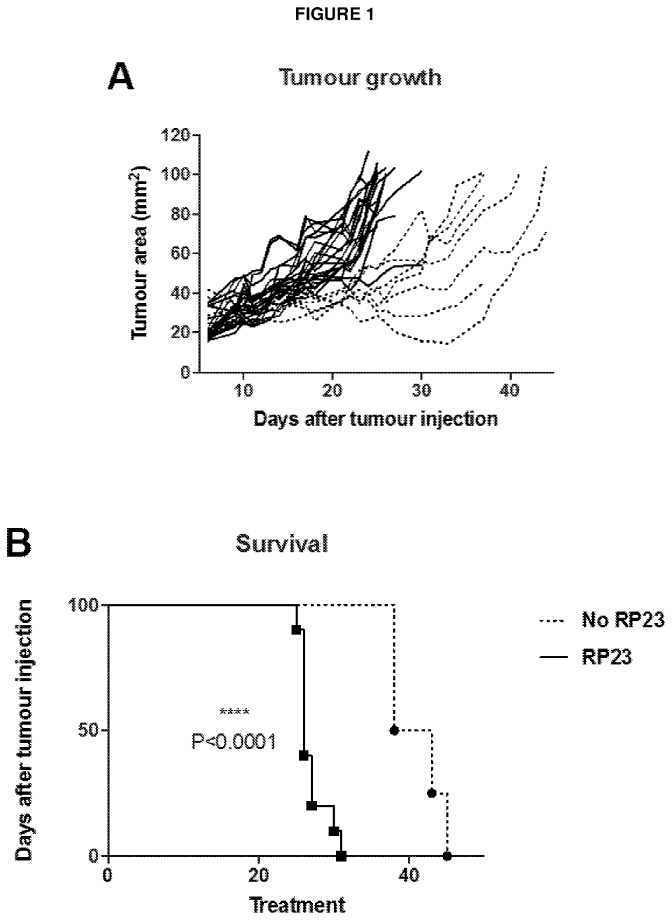

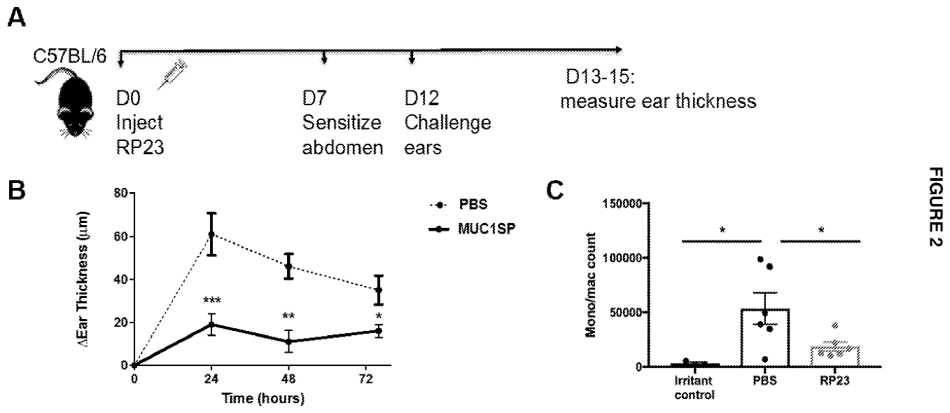

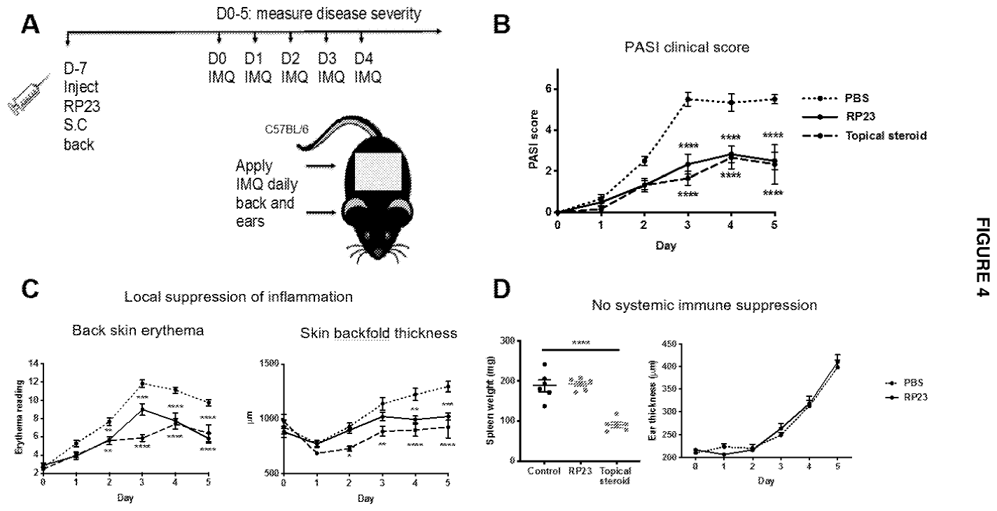

The inventors initially trialled RP23 (SEQ ID NO: 12) as a vaccine antigen in a murine skin tumour model. To their surprise, they did not observe reduced skin tumour growth in mice treated with RP23, but rather a modest increase in tumour growth, suggestive of an immune suppressive effect (FIG. 1). They then assessed whether RP23 could reduce inflammation in a murine model of contact dermatitis, using a contact hypersensitivity assay (FIG. 2a). A single nanomolar injection of RP23 prior to sensitisation was able to significantly suppress elicitation of inflammation, as measured by a reduction in ear thickness (FIG. 2b) and inflammatory myeloid cell skin infiltrate (FIG. 2c). This immune suppression was specific to the RP23 sequence, as a peptide bearing the same amino acids in a scrambled order did not significantly reduce ear swelling (FIG. 3a). RP23 considerably increased the synthetic yield without significantly altering immune suppression when compared to the effect of SEQ ID NO: 3 (MUC1SP) (FIG. 3b). Using RP23, the inventors carried out a dose-response assay to titrate the immune suppression. Using three doses in the low nanomole range (2, 5 or 10 nmol, about 0.3 0.8, 1.6 mg/kg), a dose-dependent reduction in ear swelling of up to 100% compared to controls was observed (FIG. 3c). Including GM-CSF in the assay abrogated much of the suppression afforded by RP23 (FIG. 3d). Further, in a murine model of imiquimod-induced psoriasis (FIG. 4a), a single injection of RP23 reduced local erythema, skin thickness and scaling (FIG. 4b,c). This immunomodulatory effect was isolated to the area in which RP23 was delivered, unlike topical application of a glucocorticoid which caused unwanted systemic immune suppression (FIG. 4d). No adverse reactions of any kind have been observed in mice receiving RP23 injection, even when a 10 nmol dose was given daily for 6 days by sub-cutaneous injection (data not shown), supportive of this peptide being non-toxic and non-irritating.

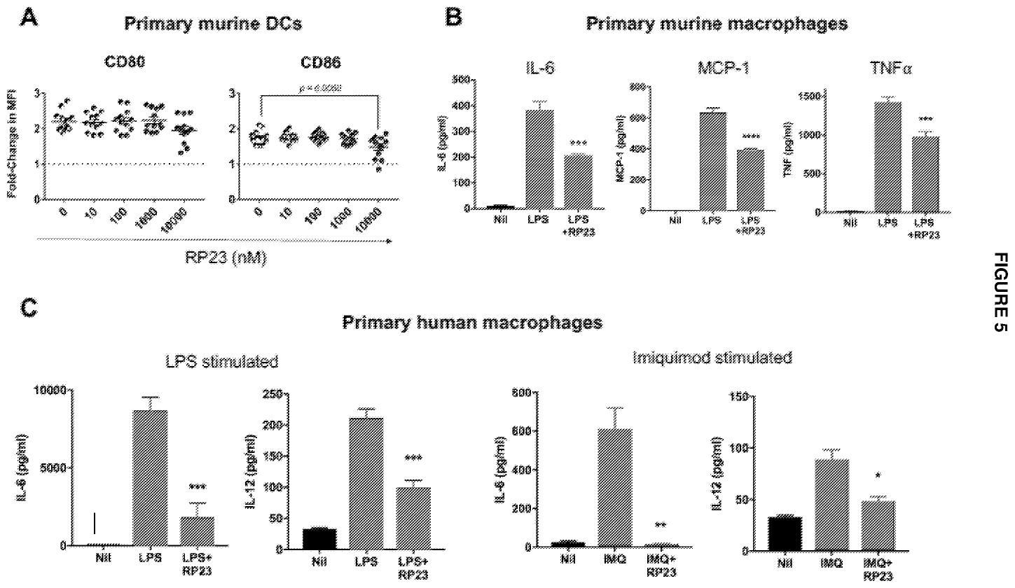

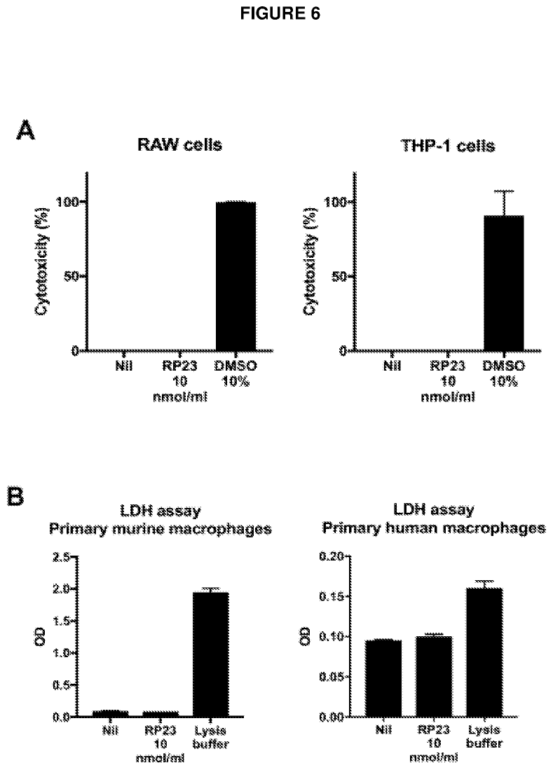

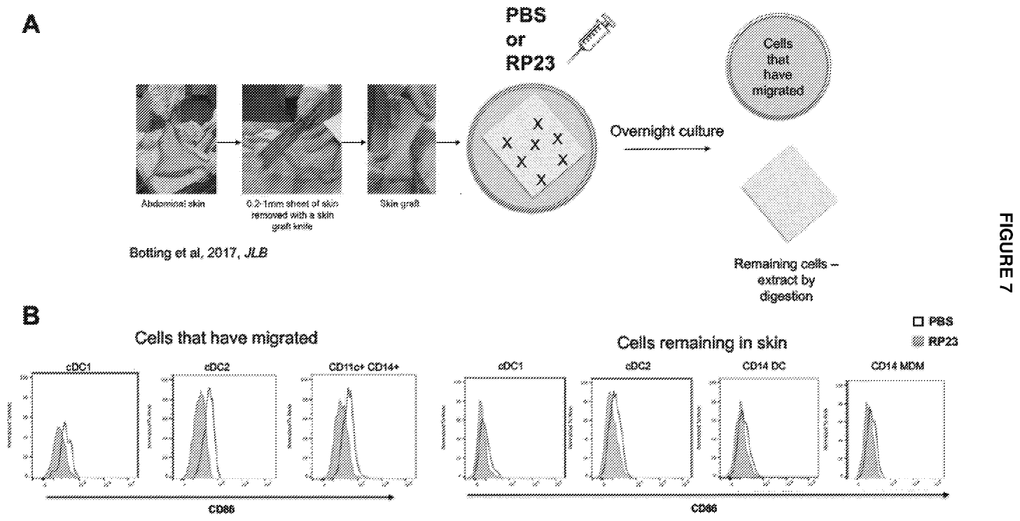

Using an in vitro culture system, it was determined whether RP23 could suppress the activity of specific pro-inflammatory immune cells. In primary murine splenic dendritic cells, RP23 reduced the secretion of inflammatory chemokines (data not shown) and expression of surface activation markers (FIG. 5a). The culture of murine bone marrow derived macrophages with RP23 led to reduced IL-6, MCP-1 and TNF release after LPS-stimulation (FIG. 5b). Similarly, primary human blood derived macrophages had reduced IL-6 and IL-12/23 release after stimulation with either LPS or imiquimod (FIG. 5c). RP23 was able to induce these changes in macrophages without causing any cytostatic or cytotoxic effects to the cells (FIG. 6), indicating that the peptide is non-toxic. When RP23 was injected intra-dermally into healthy human skin explants it reduced spontaneous activation of dermal dendritic cells (FIG. 7).

In conclusion, the peptides described herein offer potential as a novel, locally acting peptide-based therapy for patients seeking improved management of inflammatory skin diseases.

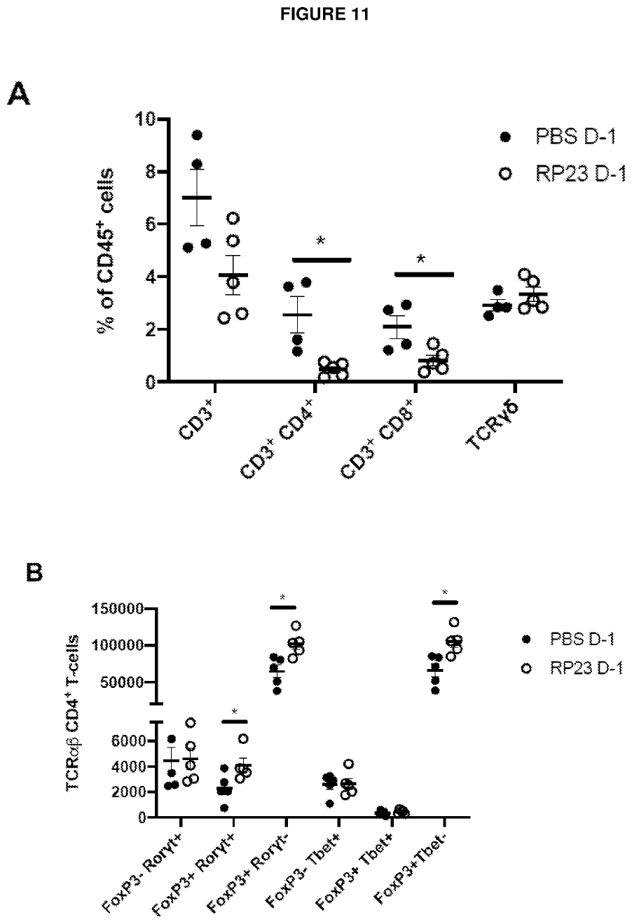

Additionally, the immune pathways modified, in particular the reduction of IL-12/23, IL-6, and TNF (FIG. 5) as well as the reduction in the activation status of pro-inflammatory immune cells such as macrophages and DCs (FIG. 5, 7, 15) suggest broader applicability of RP23 beyond inflammatory skin indications. The RP23-mediated reduction in TCRαβ+ T-cells in the skin of psoriatic mice (FIG. 11A), and the increase in FoxP3+ CD4+ T-cells in the skin draining lymph nodes (FIG. 11B), also suggest suitability for use in inflammatory conditions mediated by T-cells. This may include inflammatory conditions further characterised by T-helper 1 or 17 type inflammation, such as RA and IBD, that may be initiated by, or further aggravated via pro-inflammatory innate immune cells.

### Example 3: Effects of Variants of RP23 (SEQ ID NO:12) on the Inflammatory Response

The inventors then conducted additional in vivo studies, using a model of contact hypersensitivity, to determine the portion of RP23 responsible for its immunomodulatory activity. Mice received one injection of peptide (10 nmol) subcutaneously at the abdomen, seven days prior to sensitisation of the skin with a contact irritant (DNFB). Five days later, mice were challenged with DNFB at a distal site, on the ears. The thickness of the ears was measured over 72 hours. The thickness is proportional to the degree of the inflammatory response.

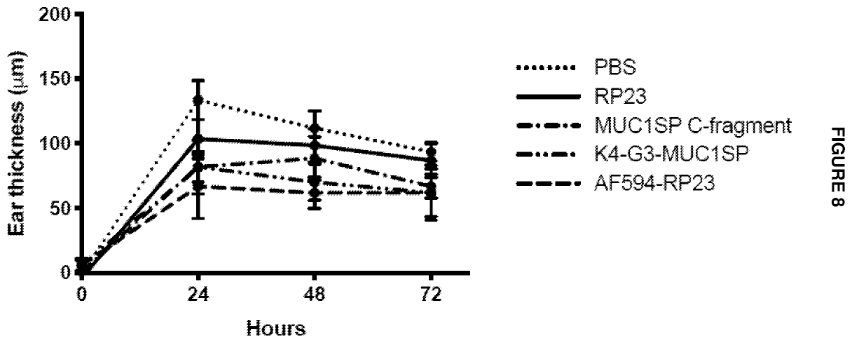

Compared to mice injected with saline (PBS), it was found that SEQ ID NO:12 (RP23), a fluorescently labelled version of SEQ ID NO:12 (SEQ ID NO:21=AF594-RP23), a version of SEQ ID NO:12 that could be produced recombinantly (SEQ ID NO:22=K4G3MUC1SP), and the C-terminal fragment of the peptide (SEQ ID NO:2=MUC1SP C-fragment) all led to a reduced inflammatory response (FIG. 8).

| Peptides | Sequence |
|---|---|
| SEQ ID NO: 12 | KKKK-PEG-MTPGTQSPFFLLLLLTVLTVV |
| SEQ ID NO: 21 | AF594-KKKK-PEG-MTPGTQSPFFLLLLLTVLTVV |
| SEQ ID NO: 22 | KKKKGGGMTPGTQSPFFLLLLLTVLTVV |
| SEQ ID NO: 2 | LLLLLTVLTVV |

### Example 4: Effects of Additional Peptide Variants on the Inflammatory Response

The effect of RP23 (SEQ ID NO:12) above was confirmed using a number of additional peptide variants labelled as SEQ ID NO: 32 and SEQ ID NO: 35 as described herein in a mouse model of contact dermatitis. The effects of these peptides were compared to a scrambled version of SEQ ID NO: 35.

Mice received one injection of PBS, RP23 (10 nmol) or one of the following peptide variants indicated in the below table (equivalent molar amount of RP23), subcutaneously at the abdomen.

| Peptide | Sequence |
|---|---|
| RP23 | KKKK-peg-MTPGTQSPFFLLLLLTVLTVV |
| (SEQ ID NO: 12) | |
| Gd-DOTA-RP23 | (Gd)DOTA-KKKK-peg-TPGTQSPFFLLL |
| (SEQ ID NO: 32) | LLTVLTVV (no N-term Met) |
| Biotin-RP23 | Biotin-KKKK-peg-TPGTQSPFFLLLLL* |
| (*photoLeu) | TVLTVV (no N-term Met) |
| (SEQ ID NO: 35) | |
| Biotin-RP23 | Biotin-KKKK-peg-QLTLTFVPSTLGFL* |
| (*photoLeu) | TLPVLV (no N-term Met) |
| scrambled | |

Photoleucine replaces leucine where indicated, these molecules used for photo-crosslinking studies. Seven days later, abdominal skin was sensitised with a contact irritant (DNFB) and five days later, mice were challenged with DNFB at a distal site, on the ears.

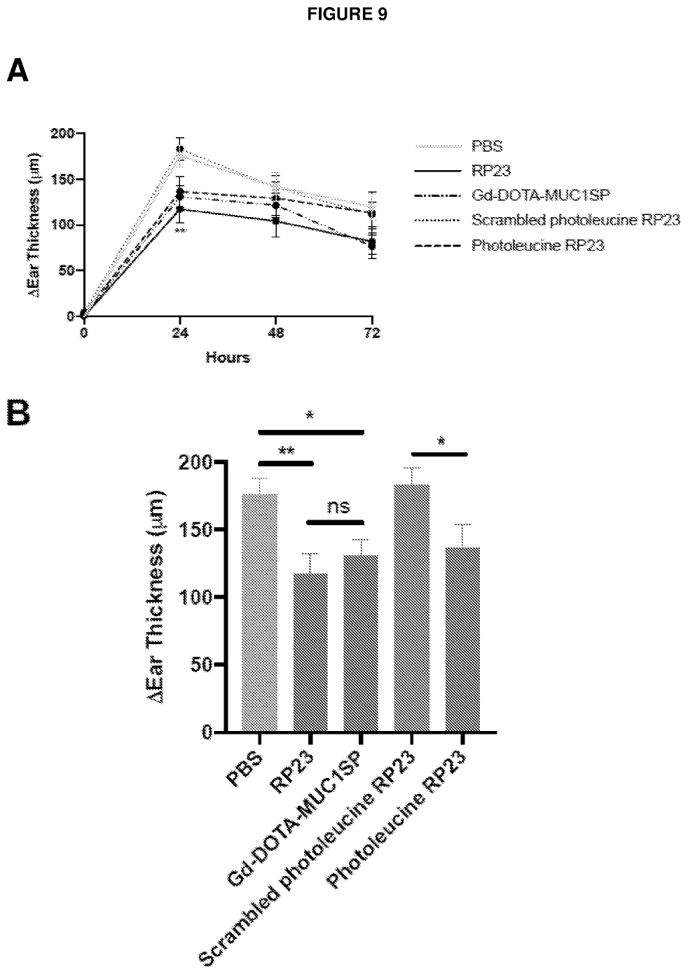

As shown in FIG. 9A the specific increase in ear thickness was reduced in mice receiving any RP23 variant where the peptide sequence is retained. In contrast, mice receiving control PBS injection, or a scrambled variant of SEQ ID NO. 35, did not have a reduction in ear inflammation. This reiterates the importance of the SEQ ID NO: 30 amino acid sequence for the immune suppressive effect, and indicates that the specified chemical modifications do not impact the efficacy of RP23. Therefore, the modified RP23 SEQ ID NOs: 32 and 35, may be used in place of RP23 for the assessment of RP23-induced immune modulation.

### Example 5: Effects of Peptides Described Herein in Combination with Corticosteroid Administration on the Inflammatory Response

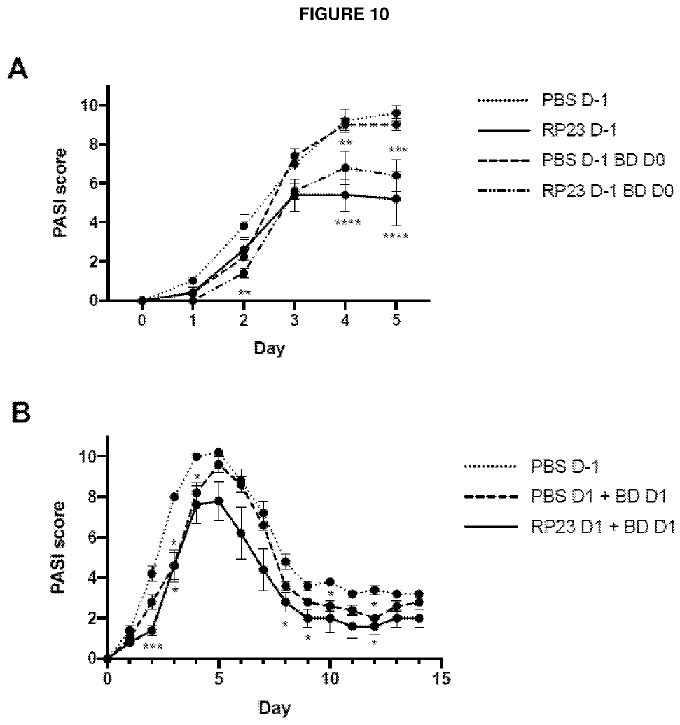

Aldara cream (5% imiquimod, 57.5 mgs) was applied daily (days 0-4) to the backs of C57BL/6 female mice to induce psoriasis. Clinical scores (PASI, modified psoriasis area and severity index) of mice receiving prophylactic PBS or RP23 (at day −1), with or without topical application of glucocorticoid (0.05% betamethasone dipropionate, 30 mgs) to the back at day 0, were determined (FIG. 10A). At early time points, that is at day 1 and 2, there were minor reductions in the clinical scores of mice receiving both RP23+BD, compared to RP23 alone or BD alone. Notably, as the disease progressed, the protective effect from BD alone was lost, whereas protection was maintained when both RP23+BD are used. However, there was no advantage of the combination compared to RP23 alone at the later time points.

RP23 and BD therapy were also tested when given synergistically at a therapeutic timepoint (at day 1). Psoriasis was induced as above, but clinical scores were determined daily for 10 days after ceasing imiquimod application at day 5, to record the rate and degree of psoriasis disease resolution (FIG. 10B). Again, RP23+BD provided an early advantage in reduction of disease (at day 2), and provided improvement compared to BD only treatment in terms of rate and degree of disease resolution. This suggests that RP23 may be used to enhance the efficacy of therapeutic BD, including when only a single administration of BD or RP23 is used.

### Example 6: RP23 Treatment Reduces TCRαβ⁺ T-Cells in Diseased Skin and Increases CD4⁺ T-Regulatory Cells in Skin Draining Lymph Nodes

C57BL/6 female mice received PBS or RP23 injection at day −1, then Aldara cream (5% imiquimod, 57.5 mgs) was applied daily (days 0-4) to the backs of mice to induce psoriasis. At day 15, skin and inguinal lymph nodes were analysed by flow cytometry to quantitate T-cell subsets as a proportion of total leukocytes (CD45+) in the psoriatic back skin, (FIG. 11A). RP23 treatment reduced the proportion of total CD4+ and CD8+ T-cells in the back skin, suggesting reduced proliferation or recruitment of pro-inflammatory T-cells to the skin in the context of psoriasis.

CD3+CD4+ TCRαβ T-cells in the skin draining lymph nodes (inguinal) were phenotyped by transcription factor staining and flow cytometry to identify cells with a regulatory phenotype (i.e., FoxP3+). In RP23 treated mice, there was a significant increase in the number of FoxP3-expressing cells, with or without the simultaneous expression of Th1 or Th17 defining transcription factors, T-bet or Rorγt respectively (FIG. 11B). This suggests that RP23 treatment promotes responses that regulate T-cell responses, including Th1 or Th17-type inflammation, supporting the utility of RP23 in any inflammatory indication where this type of inflammation is pathogenic, including psoriasis, contact dermatitis, IBD (e.g ulcerative colitis or Crohn's) and rheumatoid arthritis.

### Example 7: Peptide Formulation for Topical Application

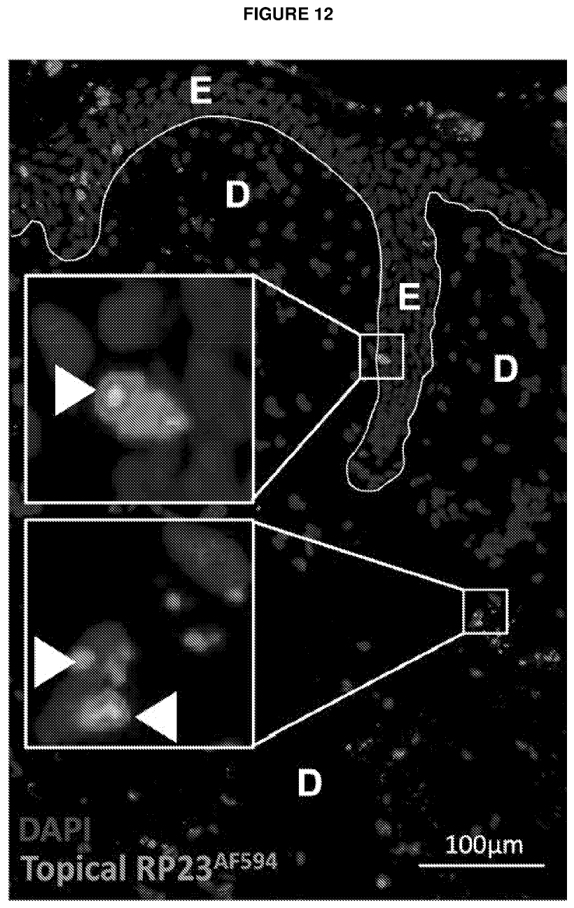

RP23^{AF594} was formulated for topical application in an adapted stock emulsion consisting of dimethicone, liquid paraffin, 4-chloro-3-methylphenol, cetrimide and octadecan-1-ol. This was applied topically (7 mg/cm², 10 nmol RP23) to full thickness human skin explants and cultured for 24 hours. Frozen sections were prepared for and analysed by fluorescence microscopy. RP23^{AF594} was detectable in both the epidermis (E) & below the basement membrane (line) in the dermis (D). This indicates that RP23 is able to penetrate the stratum corneum and basement membrane when topically applied to human skin, and is found in close association with, or is captured by, cells in both the epidermal and dermal layers of skin (FIG. 12).

### Example 8: Effect of Peptide Administration on Local Suppression of the Immune System: RP23 Treatment Reduces Inflammation in a Murine Model of Atopic Dermatitis

Ears and shaved backs of C57BL/6 female mice were sensitised (day 0) and challenged (day 7, 10, 14, 17, 19, 21, 23, 25 and 27) with topical application of the irritant oxazolone or vehicle only. Injection of mice with RP23 at day −1, or both day −7 and day −1, was compared to injection with PBS only (day −7 and day −1) or topical application of steroid (0.01% betamethasone dipropionate, 30 mgs) applied on the back at the time of every oxazolone application. Ear thickness was measured with manual callipers. Groups receiving RP23 had reduced inflammation in the ears compared to PBS injected control mice, suggesting that RP23 was reducing the priming of inflammatory responses to the topical irritant.

### Example 9: RP23 Interacts with Innate Immune Cells in the Skin and Lungs

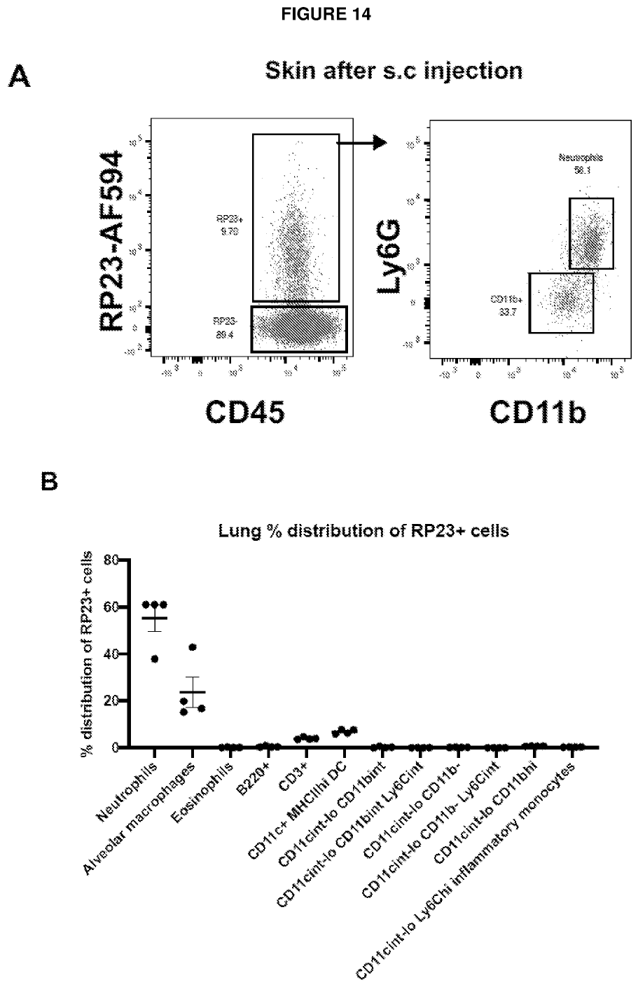

AF594-RP23+ cells were identified by flow cytometry 24 hours after administration to C57BL/6 mice. When RP23 was injected sub-cutaneously at the lower back, the peptide was primarily captured in the skin by neutrophils (CD11 b+Ly6G+) or CD11 b+ myeloid cell subsets which may include macrophages and DC (FIG. 14A). Similarly, when RP23 was delivered to the lung mucosa by intra-nasal instillation the peptide was primarily captured by neutrophils, alveolar macrophages and DC (FIG. 14B).

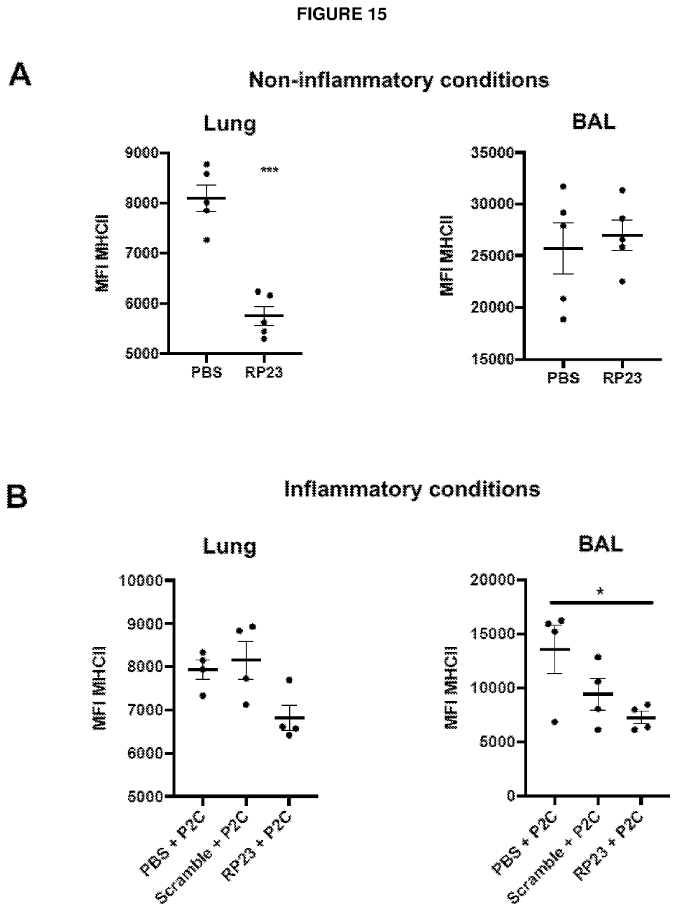

Mucosal delivery of RP23 also decreased the expression of activation markers on antigen presenting cells in the lungs. Naïve C57BL/6 mice (n=5) received either PBS or RP23 by intra-nasal instillation, then 24 hours later, cells from the bronchoalveolar lavage and perfused lungs were collected. The median fluorescence intensity of MHCII on CD11c+ dendritic cells was determined by flow cytometry. MHCII expression was substantially reduced on DC in the lung tissue (FIG. 15A). To determine if this effect would be maintained once potent lung inflammation was induced, C57BL/6 mice (n=4) received either PBS, scramble RP23 peptide, or RP23 by intra-nasal instillation, then 24 hours later, Pam2Cys-SK₄-PEG(OH) was given intra-nasally to induce an inflammatory state. RP23 treated mice again had reduced MHCII expression on lung DC and also on DC in the bronchoalveolar space (FIG. 15B). This suggests that RP23 is taken up by APC then modulates their function such that they are less pro-inflammatory.

1. An anti-inflammatory peptide comprising:
the amino acid sequence TVLTVV (SEQ ID NO:1), or the amino acid sequence LLLLLTVLTVV (SEQ ID NO:2) or functional variants or fragments thereof;
wherein the peptide consists of less than 21 amino acid residues.

2. (canceled)

3. An anti-inflammatory peptide comprising:
the amino acid sequence TVLTVV (SEQ ID NO:1), or the amino acid sequence LLLLLTVLTVV (SEQ ID NO:2) or functional variants or fragments thereof;
one or more further amino acids,
optionally one or more modifications for facilitating the solubilisation of the peptide in a biological fluid, tissue or formulation, facilitating the entry of the peptide into a cell, or for enabling detection of the peptide,
wherein the peptide comprises at least 15 amino acid residues, preferably from 15 to 1000 residues.

4. The anti-inflammatory peptide according to claim 3, wherein the peptide comprises or consists of the amino acid sequence of any one of SEQ ID NOs: 1, 2, 3, 30, or 50 to 63, or functional variants or fragments thereof.

5-6. (canceled)

7. The anti-inflammatory peptide according to claim 3, wherein the peptide consists of the sequence of SEQ ID NO: 3 or SEQ ID NO: 30, or is as defined in SEQ ID NO: 12, 32 or 35, or a functional variant thereof.

8. (canceled)

9. The anti-inflammatory peptide according to claim 3 wherein the peptide comprises a modification or moiety for facilitating the solubilisation of the peptide in a biological fluid, tissue or formulation.

10. The anti-inflammatory peptide according to claim 3, wherein the peptide comprises a modification or moiety for facilitating the solubilisation of the peptide in a biological fluid, tissue or formulation, wherein modification or moiety is selected from the group consisting of: one or more charged amino acid residues, a polymer or polymer fragment for increasing the solubility of the peptide and combinations thereof.

11-12. (canceled)

13. The anti-inflammatory peptide according to claim 3, wherein the peptide comprises a modification or moiety for facilitating the solubilisation of the peptide in a biological fluid, tissue or formulation, wherein the modification or moiety for facilitating solubilisation of the peptide is at the N terminus of the peptide.

14. The anti-inflammatory peptide according to claim 3, wherein the peptide comprises a moiety for facilitating the entry of the peptide into a cell.

15. (canceled)

16. The anti-inflammatory peptide according to claim 3, wherein the peptide comprises a moiety for enabling detection of the peptide.

17.-19. (canceled)

20. The peptide according to claim 3, the functional variant or fragment thereof has at least 85%, 86%, 87%, 88%, 89%, 90%, 91%, 92%, 93%, 94%, 95%, 96%, 97%, 98%, or at least 99% amino acid identity and retains the function of preventing or treating inflammation.

21-22. (canceled)

23. A pharmaceutical composition comprising an anti-inflammatory peptide according to claim 3, and a pharmaceutically acceptable carrier, diluent or excipient, or a pharmaceutically acceptable salt thereof.

24.-31. (canceled)

32. A method for the prevention or treatment of inflammation in an individual comprising administering a peptide of claim 3, or a pharmaceutical composition comprising a peptide of claim 3 to an individual, thereby preventing or treating inflammation in the individual.

33. The method according to claim 32, further comprising administering an additional anti-inflammatory therapy or agent selected from the group consisting of: a corticosteroid, an antigen binding protein, preferably a monoclonal antibody for inhibiting the activity of a pro-inflammatory molecule, a DMARD, phototherapy or any other agent for inhibiting inflammation in the individual.

34.-38. (canceled)

39. The method according to claim 32, wherein the inflammation is associated with an adaptive or innate immune response.

40. (canceled)

41. The method according to claim 32, wherein the inflammation is located in or on tissue selected from mucosal or skin tissue, such as dermal, musculoskeletal, pulmonary or enteric tissue.

42. The method according to claim 32, wherein the inflammation is a symptom of a disease or condition selected from the group consisting of an inflammatory skin disease or disorder, a musculoskeletal disease or disorder, a pulmonary disease or disorder, or an enteric disease or disorder.

43. The method according to claim 32, wherein the inflammation is associated or caused by an inflammatory disease or disorder selected from: rheumatoid arthritis, contact dermatitis, atopic dermatitis, allergic dermatitis, or psoriasis inflammatory disease or disorder selected from: rheumatoid arthritis, contact dermatitis, atopic dermatitis, allergic dermatitis, or psoriasis.

44. The method according to claim 32, wherein the method minimises one or more symptoms selected from the group consisting of erythema, skin thickness, scaling, pruritus and inflammatory oedema.

45. The method of claim 32, wherein the method comprises regulating an immune response in the individual.

46. (canceled)

47. The method according to claim 32, wherein the method minimises the release of inflammatory cytokines, preferably TNF, IL-6 and IL-12/23 and IL-17.

48.-50. (canceled)

51. The method according to claim 32, wherein the administration of the peptide or composition minimises the activation of professional antigen presenting cells, at the site of inflammation.

52.-55. (canceled)

56. The peptide of claim 3, wherein the one or more further amino acids do not define the sequence MTPGTQSPFF at a position N terminal to the sequence of SEQ ID NO: 1 or 2; or the sequence TGSGHASSTP at a position C terminal to the sequence of SEQ ID NO: 1 or 2.
